(19) United States
(12) Patent Application Publication
(10) Pub. No.: US 2022/0185889 A1
(43) Pub. Date: Jun. 16, 2022

(54) COMPOSITIONS AND METHODS FOR IMMUNOTHERAPY PROFILING

(71) Applicant: Georgia Tech Research Corporation, Atlanta, GA (US)

(72) Inventors: James Bowen, Cambridge, MA (US); Gabriel Kwong, Atlanta, GA (US); Quoc Mac, Atlanta, GA (US)

(21) Appl. No.: 17/275,117

(22) PCT Filed: Sep. 11, 2019
(86) PCT No.: PCT/US2019/050530

§ 371 Date: Feb. 10, 2022

Related U.S. Application Data
(60) Provisional application No. 62/729,470, filed on Sep. 11, 2018.

(51) Int. Cl. C07K 16/28; A61P 37/06; G01N 33/573; G01N 33/58

(52) U.S. Cl. CPC C07K 16/2818; G01N 33/582; G01N 33/573; A61P 37/06

(57) ABSTRACT

Compositions and methods for pharmacodynamic monitoring of immunotherapy are provided herein. The compositions include an immunotherapeutic agent linked to protease substrates. Upon administration, the compositions target to sites of disease where proteases are upregulated during responsive immunotherapy and subsequently cleave the attached substrates. Cleavage fragments are detected in a sample from the body and detection of the fragments is indicative of an effect of the immunotherapeutic agent.

### TECHNICAL FIELD OF THE INVENTION

This invention is generally related to immunotherapy and pharmacodynamic monitoring of immunotherapy.

### BACKGROUND OF THE INVENTION

Immunotherapies harness the immune system to treat myriad diseases such as cancer, organ transplant rejection, infectious disease, allergic disease, autoimmunity and chronic inflammation. Immunotherapies employ both the humoral and cellular arms of the immune response using therapeutic antibodies (e.g. pembrolizumab/αPD-1), cytokines (e.g. proleukin/IL-2), and cell-based therapies (e.g. Kymriah/CAR T cells). For example, emerging techniques that harness T cell immunity through adoptive transfer of engineered cells or reinvigorating endogenous anti-tumor CD8+ T cells through immune checkpoint blockade antibodies have placed immunotherapy at the forefront of cancer treatment research. Immunotherapies that dampen the T cell response through co-stimulation blockade (e.g. abatacept/CTLA-4 Ig) have also become a primary avenue of treatment research for preventing transplant rejection or treating autoimmune and chronic inflammatory disorders.

Despite the broad potential of immunotherapies, a majority of patients do not achieve clinical benefit, while others can develop immunotherapy resistance during or between treatment through poorly-understood mechanisms. Patients responding to immunotherapy often exhibit unconventional response patterns that can be misinterpreted as disease progression. The full potential benefit of immunotherapy is thus lacking, and techniques to identify biomarkers of immune responses are inadequate. Due to inadequacies in technologies for response monitoring and for identifying underlying resistance mechanisms, not only do diseases persist in the population, but drug development and clinical trials face significant obstacles.

Tissue biopsy remains the gold standard diagnostic but is invasive and samples less the 0.1% of the total disease site (Cyll, et al., Br J Cancer, 117(3):367-375 (2017)). Liquid biopsies offer a noninvasive approach, but biomarker dilution in blood significantly limits sensitivity (Nagrath, S., et al., Nature, 450(7173):1235-1239 (2007); Hori, et al., Sci Transl Med, 3(109):109ra16 (2011)). Imaging techniques can also be limited by low sensitivity and specificity, as well as the unconventional response patterns commonly associated with immunotherapy that can result in misidentification of responding patients as cases of treatment failure. The development of better, non-invasive biomarkers will identify responsive patients sooner and illuminate mechanisms of new immunotherapies.

Therefore, it is an object of the invention to provide immune checkpoint compositions and methods for monitoring their efficacy.

### SUMMARY OF THE INVENTION

Compositions and methods for pharmacodynamics monitoring of responses during immunotherapy are provided herein. Exemplary compositions include an immunotherapeutic agent linked to a protease substrate that senses immune cell and disease site protease activity and produces a detectable signal in the presence of protease activity. Upon administration, the compositions target to sites of disease where proteases are upregulated during responsive immunotherapy and subsequently cleave the attached substrates. Cleavage fragments are detected in a sample from the body and detection of the fragments is indicative of an effect of the immunotherapeutic agent.

In one embodiment, the therapeutic agent is an immune checkpoint inhibitor such as an anti-PD1 or anti-CTLA4 antibody. The protease substrate can also include a quencher molecule and a fluorescent molecule flanking the substrate. In one embodiment, the detectable signal is a peptide fragment of the protease.

Another embodiment provides a method of treating or preventing disease in a subject in need thereof by administering to the subject an effective amount of a therapeutic agent linked to protease substrate that provides a detectable signal in response to protease activity promoted by the therapeutic agent, detecting and measuring the signal in a sample from the subject, determining an effect of the therapeutic agent on the subject, wherein the subject is determined to be responsive to the therapeutic agent if the detectable signal is detected, and the subject is determined to be non-responsive to the therapeutic agent if the detectable signal is not detected, and administering the same effective amount of the therapeutic agent to responsive subjects, or adjusting the effective amount of therapeutic agent administered to non-responsive subjects. In one embodiment, the therapeutic agent is an immune checkpoint inhibitor such as an anti-PD1 or anti-CTLA4 antibody.

In one embodiment, a subject determined to be non-responsive to the immunotherapeutic agent is given a different immunotherapeutic agent.

In another embodiment, detecting and measuring the signal includes collecting a sample from the subject, such as a urine sample or a blood sample, and measuring the detectable signal in the sample.

### BRIEF DESCRIPTION OF THE DRAWINGS

FIG. 1 is a schematic illustration of an exemplary experimental use of the disclosed compositions and methods. Protease substrate functionalize therapeutic agents target sites of therapeutic activity, where the attached substrates are cleaved by proteases upregulated during responsive therapy, amplifying detection signals into urine. The urine sample is analyzed by mass spectrometry.

FIG. 2A is a schematic illustration of amine coupling of GranzymeB (GzmB) substrate to αPD-1 to generate “αPD-1 therasensors”. FIG. 2B is a graph showing PD-1 binding by αPD-1 modified with GzmB substrate (Therasensor) and unmodified PD-1 (αPD-1). The X-axis represents αPD-1 concentration (μg/mL; Log 10) and the Y-axis represents PD-1 binding. FIG. 2C is a flow plot of CD8 tumor infiltrating T cells showing equivalent staining with unmodified αPD-1 or αPD-1 modified with GzmB substrate (Therasensor). FIG. 2D is a graphical summary of FIG. 2C. FIG. 2E is a graph showing the protease cleavage kinetics of αPD-1 modified with GzmB substrate (Therasensor) incubated with or without GzmB or control protease thrombin.

FIG. 3A is a schematic illustration of amine coupling of GzmB substrate to CTLA-4 Ig to generate “CLTA-4 Ig therasensors”. FIGS. 3B-3C are graphs showing target binding by CTLA-4 IG modified with GzmB substrate (CTLA4-Ig Therasensor) or unmodified CTLA-4 Ig (CTLA4-Ig) in a CD80/CD86 antibody competition assay. FIG. 3D is a bar graph showing proliferation of Cell Trace Violet (CTV) labeled BL/6 CD8+ cells co-incubated with BALB/c CD11c+ dendritic cells in the presence of αCD40L only, αCD40L+ unmodified CTLA4-Ig (αCD40L+CTLA4-Ig), αCD40L+ modified CTLA4-Ig (αCD40L+ Therasensor). FIG. 3E is a line graph showing protease cleavage kinetics of CTLA-4 IG modified with GzmB substrate incubated with or without GzmB or the indicated protease (Abbreviations: CTSB, Cathepsin B; MMP2, matrix metalloproteinase 2; MMP9, matrix metalloproteinase 9; MMP15, matrix metalloproteinase 15; C1S, complement component S1; MASP1, mannose-associated serine protease 1).

FIG. 4A is a schematic illustration of the cleavage of αPD-1 modified with GzmB substrate by GzmB in activated T cells, but not in tumor cell supernatant. FIG. 4B is a line graph showing protease cleavage kinetics of αPD-1 modified with GzmB substrate (GzmB therasensor), control therasensor, or αPD-1 incubated with supernatant from activated T cells, CT26 cells, MC38 cells, B16 cells, or media alone. FIG. 4C is a schematic illustration of αPD-1 therasensor cleavage during T cell killing of tumor cells. FIG. 4D is a bar graph showing percent cytotoxicity, as measured by an LDH assay. FIG. 4E is a bar graph showing GzmB protein secretion as determined by ELISA. Increased cell killing and GzmB secretion was observed as the effector to target ratio was increased (1:1, 5:1, 10:1). FIG. 4F is a bar graph showing protease activity for control and αPD-1 therasensor across multiple ratios of effector to target cells. FIG. 4G is a bar graph showing protease activity of the αPD-1 therasensor in cells incubated with P-Mel or OT-1. FIG. 4H is a bar graph showing protease activity for CTLA-4 Ig therasensors added to supernatants from co-cultures of OT-I cells and either OVA expressing EG7 cells or the parental, non-OVA expressing EL4 cell line (E:T ratios of 1:1, 5:1, and 10:1).

FIG. 5A is a line graph showing MC38 syngeneic tumor volume over time in mice treated with αPD-1 modified with GzmB substrate (αPD-1 therasensor) or isotype control therasensor. FIG. 5B is a panel of flow cytometry plots showing intracellular GzmB staining within CD8+ TILs isolated from MC38 tumors after two treatment doses. FIGS. 5C and 5D are graphs showing the percentage (FIG. 5C) and number (FIG. 5D) of GzmB positive CD8 TILs per tumor. FIG. 5E is a schematic illustration of the experimental method for urinalysis of therasensors in MC38 tumor bearing mice. FIG. 5F is a graph showing renal clearance of peptide fragments in tumor bearing mice treated with control therasensor or α-PD1 therasensor. FIGS. 5G-5H are graphs showing tumor volume over time in CT26 tumor bearing mice treated with a-CTLA4 monotherapy (FIG. 5G), α-PD1/CTLA-4 combination therapy (FIG. 5H) or untreated. The X-axis represents time (days) and the Y-axis represents tumor volume (mm²). The gray area represents the treatment window. FIG. 5I is a panel of flow cytometry plots showing intracellular GzmB staining within CD8+ TILs isolated from CT26 tumors on day 18. FIG. 5J-5K are graphs showing the percentage (FIG. 5J) and number (FIG. 5K) of GzmB positive CD8 TILs per tumor. FIG. 5L is a schematic illustration of the experimental method for urinalysis of therasensors in CT26 tumor bearing mice. FIGS. 5M-5N are graphs showing renal clearance of cleaved fluorescent reporters in urine of tumor bearing mice treated with αCTLA-4, αPD-1/CTLA-4, or untreated.

FIG. 6A is a timeline showing the experimental procedures. FIG. 6B-6I are photos showing allograft rejection in skin over time. FIG. 6J is a plot of immunohistochemistry data showing percent of CD8 staining in graft and healthy skin tissues from mice bearing allo- and iso-grafts. FIG. 6K is a plot of immunohistochemistry data showing percent of GzmB staining in graft and healthy skin tissues from mice bearing allo- and iso-grafts. FIG. 6L is a plot of skin graft scores showing graft quality of skin allograft in untreated mice, treated mice responding weakly (“non-responding”) or strongly (“responding”) to co-stimulation blockade therapy with CTLA4-Ig and αCD154. FIG. 6I is a graft survival curve showing percent survival of grafts in untreated, non-responding, and responding grafts. FIG. 6J is a graph showing percent renal clearance of cleaved fluorescent reporters in urine at POD-4, 7, and 15.

FIG. 7A is a schematic of the patient cohort from Riaz, et al., 2017. FIG. 7B is a graph classifying responders from non-responders using 250 extracellular proteases. FIG. 7C is a graph classifying responders from non-responders using 14 extracellular proteases identified as important by lasso algorithm. FIG. 7D is a graph showing the relative weights of importance of the 14 extracellular proteases from FIG. 7C. FIG. 7E-7F are graphs identifying mechanisms of resistance via pathway analysis. FIG. 7E shows non-responding patients with IFNγ pathway expression loss were predicted with a panel of 12 proteases. FIG. 7F shows the same panel of 12 proteases was used to classify non-responding patients with MHC I antigen presentation loss. FIG. 7G is a graph showing the fraction of pathways from each molecular process (IFNγ and MHC I antigen presentation) lost when comparing gene expression of responders and non-responders. FIG. 7H is a graph showing the relative weight of lasso coefficients in classifying non-responders with or without MHC I presentation loss.

### DETAILED DESCRIPTION OF THE INVENTION

### I. Definitions

It should be appreciated that this disclosure is not limited to the compositions and methods described herein as well as the experimental conditions described, as such may vary. It is also to be understood that the terminology used herein is for the purpose of describing certain embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any compositions, methods and materials similar or equivalent to those described herein can be used in the practice or testing of the present invention. All publications mentioned are incorporated herein by reference in their entirety.

The use of the terms “a,” “an,” “the,” and similar referents in the context of describing the presently claimed invention (especially in the context of the claims) are to be construed to cover both the singular and the plural, unless otherwise indicated herein or clearly contradicted by context.

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approx. +/−10%; in other embodiments the values may range in value either above or below the stated value in a range of approx. +/−5%; in other embodiments the values may range in value either above or below the stated value in a range of approx. +/−2%; in other embodiments the values may range in value either above or below the stated value in a range of approx. +/−1%. The preceding ranges are intended to be made clear by context, and no further limitation is implied. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

As used herein, a molecule is said to be able to “immunospecifically bind” a second molecule if such binding exhibits the specificity and affinity of an antibody to its cognate antigen. Antibodies are said to be capable of immunospecifically binding to a target region or conformation (“epitope”) of an antigen if such binding involves the antigen recognition site of the immunoglobulin molecule. An antibody that immunospecifically binds to a particular antigen may bind to other antigens with lower affinity if the other antigen has some sequence or conformational similarity that is recognized by the antigen recognition site as determined by, e.g., immunoassays, BIACORE® assays, or other assays known in the art, but would not bind to a totally unrelated antigen. In some embodiments, however, antibodies (and their antigen binding fragments) will not cross-react with other antigens. Antibodies may also bind to other molecules in a way that is not immunospecific, such as to FcR receptors, by virtue of binding domains in other regions/domains of the molecule that do not involve the antigen recognition site, such as the Fc region.

As used herein, the term “antibody” is intended to denote an immunoglobulin molecule that possesses a “variable region” antigen recognition site and include antigen-binding fragments of antibodies. The term “variable region” is intended to distinguish such domain of the immunoglobulin from domains that are broadly shared by antibodies (such as an antibody Fc domain). The variable region includes a “hypervariable region” whose residues are responsible for antigen binding. The hypervariable region includes amino acid residues from a “Complementarity Determining Region” or “CDR” (i.e., typically at approximately residues 24-34 (L1), 50-56 (L2) and 89-97 (L3) in the light chain variable domain and at approximately residues 27-35 (H1), 50-65 (H2) and 95-102 (H3) in the heavy chain variable domain; Kabat et al., Sequences of Proteins of Immunological Interest, 5th Ed. Public Health Service, National Institutes of Health, Bethesda, Md. (1991)) and/or those residues from a “hypervariable loop” (i.e., residues 26-32 (L1), 50-52 (L2) and 91-96 (L3) in the light chain variable domain and 26-32 (H1), 53-55 (H2) and 96-101 (H3) in the heavy chain variable domain; Chothia and Lesk, 1987, J Mol. Biol. 196:901-917). “Framework Region” or “FR” residues are those variable domain residues other than the hypervariable region residues as herein defined. The term antibody includes monoclonal antibodies, multi-specific antibodies, human antibodies, humanized antibodies, synthetic antibodies, chimeric antibodies, camelized antibodies (See e.g., Muyldermans et al., 2001, Trends Biochem. Sci. 26:230; Nuttall et al., 2000, Cur. Pharm. Biotech. 1:253; Reichmann and Muyldermans, 1999, J. Immunol. Meth. 231:25; International Publication Nos. WO 94/04678 and WO 94/25591; U.S. Pat. No. 6,005,079), single-chain Fvs (scFv) (see, e.g., see Pluckthun in The Pharmacology of Monoclonal Antibodies, vol. 113, Rosenburg and Moore eds. Springer-Verlag, New York, pp. 269-315 (1994)), single chain antibodies, disulfide-linked Fvs (sdFv), intrabodies, diabodies, triabodies, tetrabodies, Bis-scFv, minibodies, Fab2, Fab3 and anti-idiotypic (anti-Id) antibodies (including, e.g., anti-Id and anti-anti-Id antibodies to antibodies). In particular, such antibodies include immunoglobulin molecules of any type (e.g., IgG, IgE, IgM, IgD, IgA and IgY), class (e.g., IgG₁, IgG₂, IgG₃, IgG₄, IgA₁ and IgA₂) or subclass.

As used herein, the term “antigen binding fragment” of an antibody refers to one or more portions of an antibody that contain the antibody's Complementarity Determining Regions (“CDRs”) and optionally the framework residues that include the antibody's “variable region” antigen recognition site, and exhibit an ability to immunospecifically bind antigen. Such fragments include Fab′, F(ab′)₂, Fv, single chain (ScFv), and mutants thereof, naturally occurring variants, and fusion proteins including the antibody's “variable region” antigen recognition site and a heterologous protein (e.g., a toxin, an antigen recognition site for a different antigen, an enzyme, a receptor or receptor ligand, etc.).

As used herein, the term “fragment” refers to a peptide or polypeptide including an amino acid sequence of at least 5 contiguous amino acid residues, at least 10 contiguous amino acid residues, at least 15 contiguous amino acid residues, at least 20 contiguous amino acid residues, at least 25 contiguous amino acid residues, at least 40 contiguous amino acid residues, at least 50 contiguous amino acid residues, at least 60 contiguous amino residues, at least 70 contiguous amino acid residues, at least 80 contiguous amino acid residues, at least 90 contiguous amino acid residues, at least 100 contiguous amino acid residues, at least 125 contiguous amino acid residues, at least 150 contiguous amino acid residues, at least 175 contiguous amino acid residues, at least 200 contiguous amino acid residues, or at least 250 contiguous amino acid residues.

As used herein the term “modulate” relates to a capacity to alter an effect, result, or activity (e.g., signal transduction). Such modulation can be agonistic or antagonistic. Antagonistic modulation can be partial (i.e., attenuating, but not abolishing) or it can completely abolish such activity (e.g., neutralizing). Modulation can include internalization of a receptor following binding of an antibody or a reduction in expression of a receptor on the target cell. Agonistic modulation can enhance or otherwise increase or enhance an activity (e.g., signal transduction). In a still further embodiment, such modulation can alter the nature of the interaction between a ligand and its cognate receptor so as to alter the nature of the elicited signal transduction. For example, the molecules can, by binding to the ligand or receptor, alter the ability of such molecules to bind to other ligands or receptors and thereby alter their overall activity. In some embodiments, such modulation will provide at least a 10% change in a measurable immune system activity, at least a 50% change in such activity, or at least a 2-fold, 5-fold, 10-fold, or at least a 100-fold change in such activity.

As used herein, the term “polypeptide” refers to a chain of amino acids of any length, regardless of modification (e.g., phosphorylation or glycosylation). The term polypeptide includes proteins and fragments thereof. The polypeptides can be “exogenous,” meaning that they are “heterologous,” i.e., foreign to the host cell being utilized, such as human polypeptide produced by a bacterial cell. Polypeptides are disclosed herein as amino acid residue sequences. Those sequences are written left to right in the direction from the amino to the carboxy terminus. In accordance with standard nomenclature, amino acid residue sequences are denominated by either a three letter or a single letter code as indicated as follows: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V).

As used herein, the terms “treat,” “treating,” “treatment” and “therapeutic use” refer to the elimination, reduction or amelioration of one or more symptoms of a disease or disorder. As used herein, a “therapeutically effective amount” refers to that amount of a therapeutic agent sufficient to mediate a clinically relevant elimination, reduction or amelioration of such symptoms. An effect is clinically relevant if its magnitude is sufficient to impact the health or prognosis of a recipient subject. A therapeutically effective amount may refer to the amount of therapeutic agent sufficient to delay or minimize the onset of disease, e.g., delay or minimize the spread of cancer. A therapeutically effective amount may also refer to the amount of the therapeutic agent that provides a therapeutic benefit in the treatment or management of a disease.

As used herein, the term “prophylactic agent” refers to an agent that can be used in the prevention of a disorder or disease prior to the detection of any symptoms of such disorder or disease. A “prophylactically effective” amount is the amount of prophylactic agent sufficient to mediate such protection. A prophylactically effective amount may also refer to the amount of the prophylactic agent that provides a prophylactic benefit in the prevention of disease.

As used herein, the terms “immunologic,” “immunological” or “immune” response is the development of a beneficial humoral (antibody mediated) and/or a cellular (mediated by antigen-specific T cells or their secretion products) response directed against a peptide in a recipient patient. Such a response can be an active response induced by administration of immunogen or a passive response induced by administration of antibody or primed T-cells. A cellular immune response is elicited by the presentation of polypeptide epitopes in association with Class I or Class II MEW molecules to activate antigen-specific CD4⁺ T helper cells and/or CD8⁺ cytotoxic T cells. The response may also involve activation of monocytes, macrophages, NK cells, basophils, dendritic cells, astrocytes, microglia cells, eosinophils, activation or recruitment of neutrophils or other components of innate immunity. The presence of a cell-mediated immunological response can be determined by proliferation assays (CD4⁺ T cells) or CTL (cytotoxic T lymphocyte) assays. The relative contributions of humoral and cellular responses to the protective or therapeutic effect of an immunogen can be distinguished by separately isolating antibodies and T-cells from an immunized syngeneic animal and measuring protective or therapeutic effect in a second subject.

Activated T cells that are specific to molecular structures on an invading pathogen proliferate and attack the invading pathogen. Their attack can kill pathogens directly or secrete antibodies that enhance the phagocytosis of pathogens and disrupt the infection. Some T cells respond to APCs of the innate immune system, and indirectly induce immune responses by releasing or cytokines.

As used herein, an “immune cell” refers to any cell from the hemopoietic origin including, but not limited to, T cells, B cells, monocytes, dendritic cells, and macrophages.

As used herein, “inflammatory molecules” refer to molecules that result in inflammatory responses including, but not limited to, cytokines and metalloproteases such as including, but not limited to, IL-1β, TNF-α, TGF-beta, IFN-γ, IL-18, IL-17, IL-6, IL-23, IL-22, IL-21, and MMPs.

As used herein, the terms “individual,” “host,” “subject,” and “patient” are used interchangeably herein, and refer to a mammal, including, but not limited to, humans, rodents, such as mice and rats, and other laboratory animals.

As used herein, the term “pharmaceutically acceptable carrier” encompasses any of the standard pharmaceutical carriers, such as a phosphate buffered saline solution, water and emulsions such as an oil/water or water/oil emulsion, and various types of wetting agents.

As used herein, the term “immunosuppression” refers to the suppression of the immune system and its ability to fight infections and other diseases. Immunosuppression may be deliberately induced with drugs, or it can result from certain diseases, environmental factors, or as a side effect to other drugs such as anticancer drugs and steroids.

As used herein, the term “immunosuppressive disease” and “immunodeficiency disease” refer to diseases characterized by the partial or complete suppression or dysfunction of the immune response of a subject.

As used herein, the term “cancer” refers to a neoplasm or tumor resulting from abnormal uncontrolled growth of cells. As used herein, cancer explicitly includes leukemias and lymphomas. The term “cancer” refers to a disease involving cells that have the potential to metastasize to distal sites and exhibit phenotypic traits that differ from those of non-cancer cells, for example, formation of colonies in a three-dimensional substrate such as soft agar or the formation of tubular networks or web-like matrices in a three-dimensional basement membrane or extracellular matrix preparation. Non-cancer cells do not form colonies in soft agar and form distinct sphere-like structures in three-dimensional basement membrane or extracellular matrix preparations.

### II. Compositions and Methods for Immunotherapy Profiling

Immunotherapeutic compositions and methods of their use for both treating disease in a subject in need thereof and profiling the subject's immune response to the immunotherapy are provided herein. An exemplary composition includes an immunotherapeutic agent conjugated with a protease substrate that is capable of being cleaved from the immunotherapeutic agent by disease- or tissue-specific proteases. In one embodiment, if the immunotherapeutic agent reaches the disease site and imparts a therapeutic effect, increased immune protease activity will cleave the attached protease substrate from the immunotherapeutic agent releasing a peptide fragment or detectable signal into circulation upon which it will be selectively filtered into the urine. The circulating cleavage fragment or detectable signal can be detected in a sample from the subject such as a blood sample or a urine sample.

A. Immunotherapeutic Agent

In one embodiment, the immunotherapeutic agent that is conjugated with a protease substrate is a checkpoint inhibitor. Immune checkpoint inhibitors typically employ therapeutic antibodies, such as Pembrolizumab (αPD1) or Ipilimumab (αCTLA-4), to reverse immune suppression within the tumor microenvironment by blocking inhibitory immune checkpoint molecules, such as PD-1 (Tumeh P C, et al., Nature, 515(7528):568-71 (2014)).

In one embodiment, the immunotherapeutic agent is an antibody, antigen-binding fragment, fusion protein, or small molecule. In another embodiment, the immunotherapeutic agent is a T cell therapy, such as CAR-T cell therapy. In yet another embodiment, the immunotherapeutic agent is an immunosuppressive agent. Immunotherapeutic agent targets are described in detail below.

1. PD-1

Programmed Death-1 (PD-1) is a member of the CD28 family of receptors that delivers a negative immune response when induced on T cells. Contact between PD-1 and one of its ligands (B7-H1 or B7-DC) induces an inhibitory response that decreases T cell multiplication and/or the strength and/or duration of a T cell response. Suitable PD-1 antagonists are described in U.S. Pat. Nos. 8,114,845, 8,609,089, and 8,709,416, which are specifically incorporated by reference herein in their entities, and include compounds or agents that either bind to and block a ligand of PD-1 to interfere with or inhibit the binding of the ligand to the PD-1 receptor, or bind directly to and block the PD-1 receptor without inducing inhibitory signal transduction through the PD-1 receptor.

In some embodiments, the PD-1 receptor antagonist binds directly to the PD-1 receptor without triggering inhibitory signal transduction and also binds to a ligand of the PD-1 receptor to reduce or inhibit the ligand from triggering signal transduction through the PD-1 receptor. By reducing the number and/or amount of ligands that bind to PD-1 receptor and trigger the transduction of an inhibitory signal, fewer cells are attenuated by the negative signal delivered by PD-1 signal transduction and a more robust immune response can be achieved.

It is believed that PD-1 signaling is driven by binding to a PD-1 ligand (such as B7-H1 or B7-DC) in close proximity to a peptide antigen presented by major histocompatibility complex (MHC) (see, for example, Freeman, Proc. Natl. Acad. Sci. U.S.A, 105:10275-10276 (2008)). Therefore, proteins, antibodies or small molecules that prevent co-ligation of PD-1 and TCR on the T cell membrane are also useful PD-1 antagonists.

In some embodiments, the PD-1 receptor antagonists are small molecule antagonists or antibodies that reduce or interfere with PD-1 receptor signal transduction by binding to ligands of PD-1 or to PD-1 itself, especially where co-ligation of PD-1 with TCR does not follow such binding, thereby not triggering inhibitory signal transduction through the PD-1 receptor.

Other PD-1 antagonists contemplated by the methods of this invention include antibodies that bind to PD-1 or ligands of PD-1, and other antibodies.

Suitable anti-PD-1 antibodies include, but are not limited to, those described in the following U.S. Pat. Nos. 7,332,582, 7,488,802, 7,521,051, 7,524,498, 7,563,869, 7,981,416, 8,088,905, 8,287,856, 8,580,247, 8,728,474, 8,779,105, 9,067,999, 9,073,994, 9,084,776, 9,205,148, 9,358,289, 9,387,247, 9492539, 9492540, all of which are incorporated by reference in their entireties.

Exemplary anti-B7-H1 (also referred to as anti-PD-L1) antibodies include, but are not limited to, those described in the following U.S. Pat. Nos. 8,383,796, 9,102,725, 9,273,135, 9,393,301, and 9,580,507 all of which are specifically incorporated by reference herein in their entirety.

For anti-B7-DC (also referred to as anti-PD-L2) antibodies see U.S. Pat. Nos. 7,411,051, 7,052,694, 7,390,888, 8,188,238, and 9,255,147 all of which are specifically incorporated by reference herein in their entirety.

Other exemplary PD-1 receptor antagonists include, but are not limited to B7-DC polypeptides, including homologs and variants of these, as well as active fragments of any of the foregoing, and fusion proteins that incorporate any of these. In some embodiments, the fusion protein includes the soluble portion of B7-DC coupled to the Fc portion of an antibody, such as human IgG, and does not incorporate all or part of the transmembrane portion of human B7-DC.

The PD-1 antagonist can also be a fragment of a mammalian B7-H1, for example from mouse or primate, such as a human, wherein the fragment binds to and blocks PD-1 but does not result in inhibitory signal transduction through PD-1. The fragments can also be part of a fusion protein, for example an Ig fusion protein.

Other useful polypeptides PD-1 antagonists include those that bind to the ligands of the PD-1 receptor. These include the PD-1 receptor protein, or soluble fragments thereof, which can bind to the PD-1 ligands, such as B7-H1 or B7-DC, and prevent binding to the endogenous PD-1 receptor, thereby preventing inhibitory signal transduction. B7-H1 has also been shown to bind the protein B7.1 (Butte et al., Immunity, Vol. 27, pp. 111-122, (2007)). Such fragments also include the soluble ECD portion of the PD-1 protein that includes mutations, such as the A99L mutation, that increases binding to the natural ligands (Molnar et al., PNAS, 105:10483-10488 (2008)). B7-1 or soluble fragments thereof, which can bind to the B7-H1 ligand and prevent binding to the endogenous PD-1 receptor, thereby preventing inhibitory signal transduction, are also useful.

PD-1 and B7-H1 anti-sense nucleic acids, both DNA and RNA, as well as siRNA molecules can also be PD-1 antagonists. Such anti-sense molecules prevent expression of PD-1 on T cells as well as production of T cell ligands, such as B7-H1, PD-L1 and/or PD-L2. For example, siRNA (for example, of about 21 nucleotides in length, which is specific for the gene encoding PD-1, or encoding a PD-1 ligand, and which oligonucleotides can be readily purchased commercially) complexed with carriers, such as polyethyleneimine (see Cubillos-Ruiz et al., J. Clin. Invest. 119(8): 2231-2244 (2009), are readily taken up by cells that express PD-1 as well as ligands of PD-1 and reduce expression of these receptors and ligands to achieve a decrease in inhibitory signal transduction in T cells, thereby activating T cells.

2. CTLA4

Cytotoxic T-lymphocyte-associated protein 4 (CTLA4) is a is a protein receptor that functions as an immune checkpoint and downregulates immune responses. CTLA4 is constitutively expressed in regulatory T cells but only upregulated in conventional T cells after activation. CTLA4 transmits an inhibitory signal to T cells. In some embodiments, the immunotherapeutic agent is an antagonist of CTLA4, for example an antagonistic anti-CTLA4 antibody. An example of an anti-CTLA4 antibody contemplated for use in the methods of the invention includes an antibody as described in PCT/US2006/043690 (Fischkoff et al., WO/2007/056539).

Specific examples of an anti-CTLA4 antibody useful in the methods of the invention are Ipilimumab, a human anti-CTLA4 antibody, administered at a dose of, for example, about 10 mg/kg, and Tremelimumab a human anti-CTLA4 antibody, administered at a dose of, for example, about 15 mg/kg. See also Sammartino, et al., Clinical Kidney Journal, 3(2):135-137 (2010), published online December 2009.

In other embodiments, the antagonist is a small molecule. A series of small organic compounds have been shown to bind to the B7-1 ligand to prevent binding to CTLA4 (see Erbe et al., J. Biol. Chem., 277:7363-7368 (2002). Such small organics could be administered alone or together with an anti-CTLA4 antibody to reduce inhibitory signal transduction of T cells.

3. Other Immune Checkpoint Inhibitors

In another embodiment, the immunotherapeutic agent is an immune checkpoint inhibitor that inhibits the activity of other immune checkpoint molecules such as but not limited to B7-H3, B7-H4, BTLA, IDO, KIR, LAG3, NOX2, TIM3, VISTA, SIGLEC7, and SIGLEC9. B7-H3, also known as CD276, is an immune checkpoint molecule from the B7 family.

B7-H3 participates in the regulation of T-cell-mediated immune response. It also plays a protective role in tumor cells by inhibiting natural-killer mediated cell lysis as well as a role of marker for detection of neuroblastoma cells. It is also involved in the development of acute and chronic transplant rejection and in the regulation of lymphocytic activity at mucosal surfaces. B7-H3 immunotherapeutic agents are known in the art. Exemplary anti-B7-H4 agents include, but are not limited to, those described in the following U.S. Pat. Nos. 7,847,081, 8,802,091, and 9,371,395, all of which are specifically incorporated by reference herein in their entirety.

Indoleamine 2,3-dioxygenase (IDO), is a tryptophan catabolic enzyme with immune-inhibitory properties. IDO is known to suppress T and NK cells, generate and activate Tregs and myeloid-derived suppressor cells, and promote tumor angiogenesis. IDO immunotherapeutic agents are known in the art. Exemplary anti-IDO agents include, but are not limited to, those described in the following U.S. Pat. Nos. 7,598,287, 9,598,422, and 10,323,004, all of which are specifically incorporated by reference herein in their entirety.

Lymphocyte Activation Gene-3 (LAG3) is an inhibitory receptor on antigen activated T-cells. LAG3 delivers inhibitory signals upon binding to ligands, such as FGL1. Following TCR engagement, LAG3 associates with CD3-TCR in the immunological synapse and directly inhibits T-cell activation. LAG3 suppresses immune responses by action on Tregs as well as direct effects on CD8+ T cells. LAG3 immunotherapeutic agents are known in the art. Exemplary anti-LAG3 agents include, but are not limited to, those described in the following U.S. Pat. Nos. 10,188,730 and 10,358,495, both of which are specifically incorporated by reference herein in their entirety.

V-type immunoglobulin domain-containing suppressor of T-cell activation (VISTA) is an immunoregulatory receptor which inhibits the T-cell response. VISTA is expressed on hematopoietic cells. VISTA immunotherapeutic agents are known in the art. Exemplary anti-VISTA agents include, but are not limited to, those described in the following U.S. Pat. Nos. 9,381,244 and 10,273,301, both of which are specifically incorporated by reference herein in their entirety.

4. CAR-T Cells

Another form of immunotherapy that is contemplated for use in the disclosed compositions and methods are CAR-T cells. Chimeric antigen receptor T cells (CAR-T cells) are T cells that have been genetically engineered to produce an artificial T cell receptor. This gives the engineered T cells the ability to target a specific protein. The basis of CAR-T immunotherapy is to modify T cells to recognize cancer cells in order to more effectively target and destroy them. T cells are harvested from a subject, genetically altered to express specific T cell receptors, then the resulting CAR-T cells are infused into subjects to attack their tumors. CAR-T cells can be either derived from T cells in a subject's own blood (autologous) or derived from the T cells of another healthy donor (allogeneic). Once isolated from a subject, these T cells are genetically engineered to express a specific CAR, which programs them to target an antigen that is present on the surface of tumors. For safety, CAR-T cells are engineered to be specific to an antigen expressed on a tumor that is not expressed on healthy cells.

In one embodiment, CAR-T cells are conjugated with a protease substrate that is cleaved from the CAR-T cell by proteases that are produced when the CAR-T cell affects a diseased cell. In such an embodiment, the detection of the detached detectable signal in the urine of a subject indicates that the CAR-T cells are having an effect on the subject.

5. Immunosuppressive Agents

In another embodiment, the immunotherapeutic agent is an immunosuppressive agent. Immunosuppressive agents include, but are not limited to antibodies against other lymphocyte surface markers (e.g., CD40, alpha-4 integrin) or against cytokines), fusion proteins (e.g., CTLA-4-Ig (Orencia®), TNFR-Ig (Enbrel®)), TNF-α blockers such as Enbrel, Remicade, Cimzia and Humira, cyclophosphamide (CTX) (i.e., Endoxan®, Cytoxan®, Neosar®, Procytox®, Revimmune™), methotrexate (MTX) (i.e., Rheumatrex®, Trexall®), belimumab (i.e., Benlysta®), or other immunosuppressive drugs (e.g., cyclosporin A, FK506-like compounds, rapamycin compounds, or steroids), anti-proliferatives, cytotoxic agents, or other compounds that may assist in immunosuppression.

The immunosuppressive agent can be a CTLA-4 fusion protein, such as CTLA-4-Ig (abatacept). CTLA-4-Ig fusion proteins compete with the co-stimulatory receptor, CD28, on T cells for binding to CD80/CD86 (B7-1/B7-2) on antigen presenting cells, and thus function to inhibit T cell activation. In another embodiment, the immunosuppressive agent is a CTLA-4-Ig fusion protein known as belatacept. Belatacept contains two amino acid substitutions (L104E and A29Y) that markedly increase its avidity to CD86 in vivo. In another embodiment, the immunosuppressive agent is Maxy-4.

In another embodiment, the immunosuppressive agent is cyclophosphamide (CTX). Cyclophosphamide (the generic name for Endoxan®, Cytoxan®, Neosar®, Procytox®, Revimmune™), also known as cytophosphane, is a nitrogen mustard alkylating agent from the oxazophorines group. It is used to treat various types of cancer and some autoimmune disorders. Cyclophosphamide (CTX) is the primary drug used for diffuse proliferative glomerulonephritis in patients with renal lupus.

As used herein the term “rapamycin compound” includes the neutral tricyclic compound rapamycin, rapamycin derivatives, rapamycin analogs, and other macrolide compounds which are thought to have the same mechanism of action as rapamycin (e.g., inhibition of cytokine function). The language “rapamycin compounds” includes compounds with structural similarity to rapamycin, e.g., compounds with a similar macrocyclic structure, which have been modified to enhance their therapeutic effectiveness. Exemplary Rapamycin compounds are known in the art (See, e.g. WO95122972, WO 95116691, WO 95104738, U.S. Pat. Nos. 6,015,809; 5,989,591; 5,567,709; 5,559,112; 5,530,006; 5,484,790; 5,385,908; 5,202,332; 5,162,333; 5,780,462; 5,120,727).

The language “FK506-like compounds” includes FK506, and FK506 derivatives and analogs, e.g., compounds with structural similarity to FK506, e.g., compounds with a similar macrocyclic structure which have been modified to enhance their therapeutic effectiveness. Examples of FK506-like compounds include, for example, those described in WO 00101385. In some embodiments, the language “rapamycin compound” as used herein does not include FK506-like compounds.

B. Detectable Signal Molecule

The disclosed immunotherapeutic agents are conjugated with a protease substrate that is cleaved by proteases, releasing a peptide fragment or a detectable signal from the therapeutic agent. In some embodiments, the detection signal is the cleavage product or peptide fragment of the protease substrate itself. Upon cleavage a fragment of the protease is released into circulation and detected in urine by mass spectrometry.

In other embodiments, the detection signal is a protease substrate engineered with a quencher molecule before the cleavage site and a fluorescent reporter after the cleavage site. Upon cleavage of the protease substrate, the quencher and fluorescent reporter are separated, with the reporter being released into circulation. The fluorescent signal is detected in the urine by standard methods such as flow cytometry.

The protease substrate can be conjugated to the immunotherapeutic agent using methods known in the art. In one embodiment, the protease substrate is conjugated to the immunotherapeutic agent through the introduction of a linker that forms a covalent conjugate between the protease substrate and the immunotherapeutic agent. Exemplary reactions that can be used to link the protease substrate include but are not limited to amine-to-amine crosslinkers using NHS esters, thiol-to-thiol crosslinkers using maleimides, amine-to-thiol crosslinkers using NHS esters and maleimides, and biotin/streptavidin interactions. In one embodiment, the protease substrate is conjugated to the immunotherapeutic agent through an amine coupling reaction.

1. Protease Substrate

The disclosed compositions and methods of their use to determine the efficacy of a therapeutic response rely on protease activity to cleave the protease substrate and release a peptide fragment or detectable signal from the therapeutic agent. Proteases are a class of enzymes that includes over 550 members encoded within the human genome, many of which have disease specific roles, including critical roles in immunity. For example, cytotoxic T cell-mediated target cell killing is a protease-driven process involving: 1) death receptor signaling and caspase activation, proteases whose activity mediates cell death, and 2) secretion of granzymes, proteases that enter target cells through a perforin dependent mechanism to activate caspase-mediated cell death. Moreover, proteases are central to other aspects of immune activity including cell migration, matrix degradation and repair, and complement activation, while tumor proteases such as inflammatory and matrix degrading proteases are established hallmarks of cancer (Arias, et al., Trends Cancer, 3(6):407-422 (2017); Egeblad, et al., Nat Rev Cancer, 2(3):161-174 (2002)).

Proteases provide an innovative approach for immunotherapy response monitoring given that proteases play a central role in the underlying biology of immunity, oncology, and the pathophysiology of multiple diseases (Dudani, et al., Ann Rev of Cancer Biology, (2018)). For example, the mark of a “hot” tumor is signified by an effective immune infiltrate of cytotoxic T cells that kill cancer cells primarily through a perforin-dependent, granzyme-mediated pathway, the latter of which comprise a family of potent serine proteases (Larimer, et al., Cancer Res, 77(9):2318-2327 (2017); Voskoboinik, et al., Nat Rev Immunol, 15(6):388-400 (2015)). Tumor expression of proteases, including inflammatory and matrix degrading proteases, is well established as a hallmark of fundamental tumor biology including angiogenesis, growth, and metastasis (Dudani, et al., Ann Rev of Cancer Biology, (2018)). These protease signatures can be used to stage cancer, monitor progression and regression, and provide early indication of drug response. In one embodiment, the disclosed immunotherapeutic agents have the ability to quantify the activity of immune and disease site specific proteases early in treatment to allow identification of activity biomarkers that predict treatment efficacy and indicate resistance to immunotherapy.

In one embodiment, catalytic proteases amplify detection signals at the disease or therapeutic site (×1000 fold). Following protease cleavage, the immunotherapeutic agents disclosed herein are concentrated into urine, instead of being diluted in blood, further enriching the signal up to 100-fold. This enables ultrasensitive and early detection of T cell activity that precedes radiographic detectable changes at the disease site.

Protease substrates contain a recognition sequence for the protease to cleave. Cleavage of the protease substrate conjugated to the immunotherapeutic agent releases a peptide fragment of the substrate of a detectable signal molecule linked to the substrate from the immunotherapeutic agent. In some embodiment, the protease substrates that are conjugated to the immunotherapeutic agent are tumor specific protease substrates. Exemplary tumor associated proteases include but are not limited to cathepsin B, cathepsin D, cathepsin E, cathepsin K, cathepsin L, kallikrein 1, kallikrein 3 (PSA), kallikrein 10, kallikrein15, uPA, uPAR, caspases, matrix metalloproteinases such as MMP1, MMP2, MMP8, MMP9, MMP13, MMP14, and ADAM. In another embodiment, the protease substrates are cell specific protease substrates, such as T cell specific protease substrates. Exemplary cell specific proteases include but are not limited to neutrophil serine proteases such as cathepsin G, neutrophil elastase, and proteinase 3, mucosa-associated lymphoid tissue 1 (MALT1), granzymes, and cysteine proteinases of the caspase family, such as caspase-3, -6, -7, -8.

2. Other Detection Molecules

In some embodiments, the detection signal is a protease substrate engineered with a quencher molecule before the cleavage site and a fluorophore or fluorescent reporter after the cleavage site. Quencher molecules are known in the art. Exemplary quencher molecules include but are not limited to Deep Dark Quenchers (Eurogentec), DABCYL, TAMRA, BHQ-1®, BHQ-2®, BHQ-3®, BBQ®-650, ECLIPSE, Iowa Black® quenchers, and QSY. Exemplary fluorophores or fluorescent reporters include but are not limited to 6-FAM™, TET™, JOE™, HEX™, VIC®, cyanine 3, ROX™, LC Red 640, cyanine 5, fluorescein isothiocyanate (FITC), rhodamine (tetramethyl rhodamine isothiocyanate, TRITC, Oregon Green, Pacific Blue, Pacific Green, Pacific Orange, Texas Red, Alexa Fluor 350, Alexa Fluor 405, Alexa Fluor 488, Alexa Fluor 532, Alexa Fluor 546, Alexa Fluor 555, Alexa Fluor 568, Alexa Fluor 594, Alexa Fluor 647, Alexa Fluor 680, and Alexa Fluor 750.

In some embodiment, the protease substrate is engineered with other detectable molecules such as avidin, biotin, beta-galactosidase, luciferase, alkaline phosphatase (AP), and horseradish peroxidase (HRP). In such embodiment, the detectable molecule is cleaved from the protease substrate, which stays attached to the immunotherapeutic agent, and released into circulation. The detectable molecules are then detected in urine samples using appropriate detection method such as but not limited to ELISA, Western blotting, immunoassays, and bioluminescent assays.

C. Pharmaceutical Compositions

Pharmaceutical compositions including the disclosed activity sensing immunotherapeutic agents are provided. Pharmaceutical compositions containing the immunotherapeutic agents can be for administration by parenteral (intramuscular, intraperitoneal, intravenous (IV) or subcutaneous injection), transdermal (either passively or using iontophoresis or electroporation), or transmucosal (nasal, vaginal, rectal, or sublingual) routes of administration or using bioerodible inserts and can be formulated in dosage forms appropriate for each route of administration.

In some in vivo approaches, the compositions disclosed herein are administered to a subject in a therapeutically effective amount. As used herein the term “effective amount” or “therapeutically effective amount” means a dosage sufficient to treat, inhibit, or alleviate one or more symptoms of the disorder being treated or to otherwise provide a desired pharmacologic and/or physiologic effect. The precise dosage will vary according to a variety of factors such as subject-dependent variables (e.g., age, immune system health, etc.), the disease, and the treatment being effected.

For the disclosed immunomodulatory agents, as further studies are conducted, information will emerge regarding appropriate dosage levels for treatment of various conditions in various patients, and the ordinary skilled worker, considering the therapeutic context, age, and general health of the recipient, will be able to ascertain proper dosing. The selected dosage depends upon the desired therapeutic effect, on the route of administration, and on the duration of the treatment desired. For the disclosed immunomodulatory agents, generally dosage levels of 0.001 to 20 mg/kg of body weight daily are administered to mammals. Dosages for anti-PD-1, anti-B7-H1, and anti-CTLA4 antibody, are known in the art and can be in the range of, for example, 0.1 to 100 mg/kg, or with shorter ranges of 1 to 50 mg/kg, or 10 to 20 mg/kg. An appropriate dose for a human subject can be between 5 and 15 mg/kg, with 10 mg/kg of antibody (for example, human anti-PD-1 antibody) being a specific embodiment. Generally, for intravenous injection or infusion, dosage may be lower.

In certain embodiments, the immunomodulatory agent is administered locally, for example by injection directly into a site to be treated. Typically, the injection causes an increased localized concentration of the immunomodulatory agent composition which is greater than that which can be achieved by systemic administration. The immunomodulatory agent compositions can be combined with a matrix as described above to assist in creating an increased localized concentration of the polypeptide compositions by reducing the passive diffusion of the polypeptides out of the site to be treated.

1. Formulations for Parenteral Administration

In some embodiments, compositions disclosed herein, including those containing peptides and polypeptides, are administered in an aqueous solution, by parenteral injection. The formulation may also be in the form of a suspension or emulsion. In general, pharmaceutical compositions are provided including effective amounts of a peptide or polypeptide, and optionally include pharmaceutically acceptable diluents, preservatives, solubilizers, emulsifiers, adjuvants and/or carriers. Such compositions optionally include one or more of the following: diluents, sterile water, buffered saline of various buffer content (e.g., Tris-HCl, acetate, phosphate), pH and ionic strength; and additives such as detergents and solubilizing agents (e.g., TWEEN 20 (polysorbate-20), TWEEN 80 (polysorbate-80)), anti-oxidants (e.g., ascorbic acid, sodium metabisulfite), and preservatives (e.g., Thimersol, benzyl alcohol) and bulking substances (e.g., lactose, mannitol). Examples of non-aqueous solvents or vehicles are propylene glycol, polyethylene glycol, vegetable oils, such as olive oil and corn oil, gelatin, and injectable organic esters such as ethyl oleate. The formulations may be lyophilized and redissolved/resuspended immediately before use. The formulation may be sterilized by, for example, filtration through a bacteria retaining filter, by incorporating sterilizing agents into the compositions, by irradiating the compositions, or by heating the compositions.

2. Formulations for Oral Administration

In embodiments the compositions are formulated for oral delivery. Oral solid dosage forms are described generally in Remington's Pharmaceutical Sciences, 18th Ed. 1990 (Mack Publishing Co. Easton Pa. 18042) at Chapter 89. Solid dosage forms include tablets, capsules, pills, troches or lozenges, cachets, pellets, powders, or granules or incorporation of the material into particulate preparations of polymeric compounds such as polylactic acid, polyglycolic acid, etc. or into liposomes. Such compositions may influence the physical state, stability, rate of in vivo release, and rate of in vivo clearance of the disclosed. See, e.g., Remington's Pharmaceutical Sciences, 18th Ed. (1990, Mack Publishing Co., Easton, Pa. 18042) pages 1435-1712 which are herein incorporated by reference. The compositions may be prepared in liquid form, or may be in dried powder (e.g., lyophilized) form. Liposomal or proteinoid encapsulation may be used to formulate the compositions. Liposomal encapsulation may be used and the liposomes may be derivatized with various polymers (e.g., U.S. Pat. No. 5,013,556). See also Marshall, K. In: Modern Pharmaceutics Edited by G. S. Banker and C. T. Rhodes Chapter 10, 1979. In general, the formulation will include the peptide (or chemically modified forms thereof) and inert ingredients which protect peptide in the stomach environment, and release of the biologically active material in the intestine.

The agents can be chemically modified so that oral delivery of the derivative is efficacious. Generally, the chemical modification contemplated is the attachment of at least one moiety to the component molecule itself, where the moiety permits uptake into the blood stream from the stomach or intestine, or uptake directly into the intestinal mucosa. Also desired is the increase in overall stability of the component or components and increase in circulation time in the body. PEGylation is an exemplary chemical modification for pharmaceutical usage. Other moieties that may be used include: propylene glycol, copolymers of ethylene glycol and propylene glycol, carboxymethyl cellulose, dextran, polyvinyl alcohol, polyvinyl pyrrolidone, polyproline, poly-1,3-dioxolane and poly-1,3,6-tioxocane [see, e.g., Abuchowski and Davis (1981) “Soluble Polymer-Enzyme Adducts,” in Enzymes as Drugs. Hocenberg and Roberts, eds. (Wiley-Interscience: New York, N.Y.) pp. 367-383; and Newmark, et al. (1982) J. Appl. Biochem. 4:185-189].

Another embodiment provides liquid dosage forms for oral administration, including pharmaceutically acceptable emulsions, solutions, suspensions, and syrups, which may contain other components including inert diluents; adjuvants such as wetting agents, emulsifying and suspending agents; and sweetening, flavoring, and perfuming agents.

Controlled release oral formulations may be desirable. The agent can be incorporated into an inert matrix which permits release by either diffusion or leaching mechanisms, e.g., gums. Slowly degenerating matrices may also be incorporated into the formulation. Another form of a controlled release is based on the Oros therapeutic system (Alza Corp.), i.e., the drug is enclosed in a semipermeable membrane which allows water to enter and push drug out through a single small opening due to osmotic effects.

For oral formulations, the location of release may be the stomach, the small intestine (the duodenum, the jejunum, or the ileum), or the large intestine. In some embodiments, the release will avoid the deleterious effects of the stomach environment, either by protection of the agent (or derivative) or by release of the agent (or derivative) beyond the stomach environment, such as in the intestine. To ensure full gastric resistance a coating impermeable to at least pH 5.0 is essential. Examples of the more common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D™, Aquateric™ cellulose acetate phthalate (CAP), Eudragit L™, Eudragit S™, and Shellac™. These coatings may be used as mixed films.

3. Formulations for Topical Administration

The disclosed immunotherapeutic agents can be applied topically. Topical administration does not work well for most peptide formulations, although it can be effective especially if applied to the lungs, nasal, oral (sublingual, buccal), vaginal, or rectal mucosa.

Compositions can be delivered to the lungs while inhaling and traverse across the lung epithelial lining to the blood stream when delivered either as an aerosol or spray dried particles having an aerodynamic diameter of less than about 5 microns.

A wide range of mechanical devices designed for pulmonary delivery of therapeutic products can be used, including but not limited to nebulizers, metered dose inhalers, and powder inhalers, all of which are familiar to those skilled in the art. Some specific examples of commercially available devices are the Ultravent nebulizer (Mallinckrodt Inc., St. Louis, Mo.); the Acorn II nebulizer (Marquest Medical Products, Englewood, Colo.); the Ventolin metered dose inhaler (Glaxo Inc., Research Triangle Park, N.C.); and the Spinhaler powder inhaler (Fisons Corp., Bedford, Mass.). Nektar, Alkermes and Mannkind all have inhalable insulin powder preparations approved or in clinical trials where the technology could be applied to the formulations described herein.

Formulations for administration to the mucosa will typically be spray dried drug particles, which may be incorporated into a tablet, gel, capsule, suspension or emulsion. Standard pharmaceutical excipients are available from any formulator.

Transdermal formulations may also be prepared. These will typically be ointments, lotions, sprays, or patches, all of which can be prepared using standard technology. Transdermal formulations may require the inclusion of penetration enhancers.

D. Methods of Use

The disclosed activity sensing immunotherapeutic agents are useful for the prediction and pharmacodynamic monitoring of immunotherapy responses in a subject being administered the immunotherapeutic agent for the treatment of a disease or disorder. In such an application, the subject is being treated for a disease or disorder and being non-invasively monitored for a response to the treatment using a singular composition. In one embodiment, the subject is administered the immunotherapeutic agent or a composition including the immunotherapeutic agent. After a period of time, a sample is obtained from the subject. The sample can be blood or urine. The sample is analyzed for the presence of the detectable signal associated with the immunotherapeutic agent. In one embodiment, the detectable signal is analyzed by ELISA, mass spectrometry, flow cytometry, colorimetric analysis, bioluminescence, or immunoassay.

In one embodiment, if the detectable signal molecule is present in the sample above a detectable limit, the subject is deemed responsive to the treatment and is administered the remainder of their therapeutic regimen at the effective dose initially administered. If the detectable signal molecule is not present in the sample, the subject is deemed non-responsive and either taken off of the therapeutic regimen, or the dose of the therapeutic regimen is increased for the next dose and the detection process is repeated. If the subject continually shows no signs of detectable signal molecule in their urine sample, the subject is taken off of the therapeutic regimen. In some embodiments, the subject is switched to a different therapeutic agent disclosed herein, or the subject is switched to a different type of therapy such as chemotherapy or CAR-T cell therapy.

In another embodiment, a plurality of immunotherapeutic agents or a composition including a plurality of immunotherapeutic agents are administered to the subject and each of the detectable signals are analyzed in the subject's urine to create a signal profile. In such an embodiment, the panel of immunotherapeutic agents can be used to differentiate mechanisms of resistance in non-responsive subjects. The disclosed immunotherapeutic agents can determine if a subject has primary resistance, or acquired resistance to the immunotherapy. In primary resistance the subject is non-responsive to the immunotherapeutic upon the initial administration of the immunotherapeutic. In some embodiments, the subject has primary resistance because of the lack of recognition by T cells because of the lack of tumor antigens. In other embodiments, the cancer cells may have tumor antigens but develop mechanisms to avoid presenting them on the surface restricted by MHC.

Acquired resistance is resistance to an immunotherapeutic upon subsequent administration of the immunotherapeutic. In some embodiments, acquired resistance occurs because of loss of T cell function, lack of T cell recognition by downregulation of tumor antigen presentation, and development of escape mutation variants in the cancer. In one embodiment, panels of immunotherapeutic agents are constructed in which the expression patterns can classify subjects into different classes of resistance to the immunotherapeutic agent. Common mechanisms of immunotherapy resistance include but are not limited to loss of sensitivity to IFN-γ, loss of expression of receptors on MHC, co-expression of inhibitory receptors, upregulation of alternate inhibitory checkpoints, and high mutation overload in tumors.

In one embodiment, cancer resistance proteases are known in the art and panels of such proteases can be used to classify resistance. In one embodiment, resistance due to loss of signaling through IFN-γ can be determined using a panel of immunotherapeutics having conjugated protease substrates including but not limited to all or some of GZMA, PRSS55, PRSS48, KLK15, MMP21, CPA1, MMP23A, CTRB1, MMP24, PRSS3P2, TPSG1, OVCH2, PHEX, and KLK14. In another embodiment, resistance due to loss of beta-2-microglobulin (B2M) expression on MHC I can be determined using a panel of immunotherapeutics having conjugated protease substrates including but not limited to all or some of PLAU, ADAMS, CELA2B, CASP4, CPD, MMP25, MME, NUP98, CPLD, ASTL, ECE1, and USP32.

1. Subjects to be Treated

a. Cancer

The disclosed compositions and methods can be used to treat cancer. Generally, the agents are used to stimulate or enhance an immune response to cancer in the subject by administering to the subject an amount of the disclosed activity sensing immunotherapeutic agent. The immunotherapeutic agent can bind an inhibitory immune checkpoint molecule or its receptor and promote or enhance an immune response by inhibiting signal transduction through the immune checkpoint molecule. The method can reduce one or more symptoms of the cancer.

In one embodiment, the disclosed immunotherapeutic agents reverse immune suppression within the tumor microenvironment by blocking inhibitory immune checkpoint molecules.

Cancer cells acquire a characteristic set of functional capabilities during their development through various mechanisms. Such capabilities include evading apoptosis, self-sufficiency in growth signals, insensitivity to anti-growth signals, tissue invasion/metastasis, limitless replicative potential, and sustained angiogenesis. The term “cancer cell” is meant to encompass both pre-malignant and malignant cancer cells. In some embodiments, cancer refers to a benign tumor, which has remained localized. In other embodiments, cancer refers to a malignant tumor, which has invaded and destroyed neighboring body structures and spread to distant sites. In yet other embodiments, the cancer is associated with a specific cancer antigen (e.g., pan-carcinoma antigen (KS 1/4), ovarian carcinoma antigen (CA125), prostate specific antigen (PSA), carcinoembryonic antigen (CEA), CD19, CD20, HER2/neu, etc.).

The methods and compositions disclosed herein are useful in the treatment or prevention of a variety of cancers or other abnormal proliferative diseases, including (but not limited to) the following: carcinoma, including that of the bladder, breast, colon, kidney, liver, lung, ovary, pancreas, stomach, cervix, thyroid and skin; including squamous cell carcinoma; hematopoietic tumors of lymphoid lineage, including leukemia, acute lymphocytic leukemia, acute lymphoblastic leukemia, B-cell lymphoma, T-cell lymphoma, Berketts lymphoma; hematopoietic tumors of myeloid lineage, including acute and chronic myelogenous leukemias and promyelocytic leukemia; tumors of mesenchymal origin, including fibrosarcoma and rhabdomyoscarcoma; other tumors, including melanoma, seminoma, tetratocarcinoma, neuroblastoma and glioma; tumors of the central and peripheral nervous system, including astrocytoma, neuroblastoma, glioma, and schwannomas; tumors of mesenchymal origin, including fibrosarcoma, rhabdomyoscarama, and osteosarcoma; and other tumors, including melanoma, xenoderma pegmentosum, keratoactanthoma, seminoma, thyroid follicular cancer and teratocarcinoma.

Cancers caused by aberrations in apoptosis can also be treated by the disclosed methods and compositions. Such cancers may include, but are not be limited to, follicular lymphomas, carcinomas with p53 mutations, hormone dependent tumors of the breast, prostate and ovary, and precancerous lesions such as familial adenomatous polyposis, and myelodysplastic syndromes. In specific embodiments, malignancy or dysproliferative changes (such as metaplasias and dysplasias), or hyperproliferative disorders, are treated or prevented by the methods and compositions in the ovary, bladder, breast, colon, lung, skin, pancreas, or uterus. In other specific embodiments, sarcoma, melanoma, or leukemia is treated or prevented by the methods and compositions.

Specific cancers and related disorders that can be treated or prevented by methods and compositions disclosed herein include, but are not limited to, leukemias including, but not limited to, acute leukemia, acute lymphocytic leukemia, acute myelocytic leukemias such as myeloblastic, promyelocytic, myelomonocytic, monocytic, erythroleukemia leukemias and myelodysplastic syndrome, chronic leukemias such as but not limited to, chronic myelocytic (granulocytic) leukemia, chronic lymphocytic leukemia, hairy cell leukemia; polycythemia vera; lymphomas such as, but not limited to, Hodgkin's disease or non-Hodgkin's disease lymphomas (e.g., diffuse anaplastic lymphoma kinase (ALK) negative, large B-cell lymphoma (DLBCL); diffuse anaplastic lymphoma kinase (ALK) positive, large B-cell lymphoma (DLBCL); anaplastic lymphoma kinase (ALK) positive, ALK+ anaplastic large-cell lymphoma (ALCL), acute myeloid lymphoma (AML)); multiple myelomas such as, but not limited to, smoldering multiple myeloma, nonsecretory myeloma, osteosclerotic myeloma, plasma cell leukemia, solitary plasmacytoma and extramedullary plasmacytoma; Waldenstrom's macroglobulinemia; monoclonal gammopathy of undetermined significance; benign monoclonal gammopathy; heavy chain disease; bone and connective tissue sarcomas such as, but not limited to, bone sarcoma, osteosarcoma, chondrosarcoma, Ewing's sarcoma, malignant giant cell tumor, fibrosarcoma of bone, chordoma, periosteal sarcoma, soft-tissue sarcomas, angiosarcoma (hemangiosarcoma), fibrosarcoma, Kaposi's sarcoma, leiomyosarcoma, liposarcoma, lymphangiosarcoma, neurilemmoma, rhabdomyosarcoma, synovial sarcoma; brain tumors including but not limited to, glioma, astrocytoma, brain stem glioma, ependymoma, oligodendroglioma, nonglial tumor, acoustic neurinoma, craniopharyngioma, medulloblastoma, meningioma, pineocytoma, pineoblastoma, primary brain lymphoma; breast cancer including, but not limited to, adenocarcinoma, lobular (small cell) carcinoma, intraductal carcinoma, medullary breast cancer, mucinous breast cancer, tubular breast cancer, papillary breast cancer, Paget's disease, and inflammatory breast cancer; adrenal cancer, including but not limited to, pheochromocytom and adrenocortical carcinoma; thyroid cancer such as but not limited to papillary or follicular thyroid cancer, medullary thyroid cancer and anaplastic thyroid cancer; pancreatic cancer, including but not limited to, insulinoma, gastrinoma, glucagonoma, vipoma, somatostatin-secreting tumor, and carcinoid or islet cell tumor; pituitary cancers including but not limited to, Cushing's disease, prolactin-secreting tumor, acromegaly, and diabetes insipius; eye cancers including, but not limited to, ocular melanoma such as iris melanoma, choroidal melanoma, and cilliary body melanoma, and retinoblastoma; vaginal cancers, including, but not limited to, squamous cell carcinoma, adenocarcinoma, and melanoma; vulvar cancer, including but not limited to, squamous cell carcinoma, melanoma, adenocarcinoma, basal cell carcinoma, sarcoma, and Paget's disease; cervical cancers including, but not limited to, squamous cell carcinoma, and adenocarcinoma; uterine cancers including, but not limited to, endometrial carcinoma and uterine sarcoma; ovarian cancers including, but not limited to, ovarian epithelial carcinoma, borderline tumor, germ cell tumor, and stromal tumor; esophageal cancers including, but not limited to, squamous cancer, adenocarcinoma, adenoid cyctic carcinoma, mucoepidermoid carcinoma, adenosquamous carcinoma, sarcoma, melanoma, plasmacytoma, verrucous carcinoma, and oat cell (small cell) carcinoma; stomach cancers including, but not limited to, adenocarcinoma, fungating (polypoid), ulcerating, superficial spreading, diffusely spreading, malignant lymphoma, liposarcoma, fibrosarcoma, and carcinosarcoma; colon cancers; rectal cancers; liver cancers including, but not limited to, hepatocellular carcinoma and hepatoblastoma, gallbladder cancers including, but not limited to, adenocarcinoma; cholangiocarcinomas including, but not limited to, papillary, nodular, and diffuse; lung cancers including but not limited to, non-small cell lung cancer, squamous cell carcinoma (epidermoid carcinoma), adenocarcinoma, large-cell carcinoma and small-cell lung cancer; testicular cancers including, but not limited to, germinal tumor, seminoma, anaplastic, classic (typical), spermatocytic, nonseminoma, embryonal carcinoma, teratoma carcinoma, choriocarcinoma (yolk-sac tumor), prostate cancers including, but not limited to, adenocarcinoma, leiomyosarcoma, and rhabdomyosarcoma; penal cancers; oral cancers including, but not limited to, squamous cell carcinoma; basal cancers; salivary gland cancers including, but not limited to, adenocarcinoma, mucoepidermoid carcinoma, and adenoidcystic carcinoma; pharynx cancers including, but not limited to, squamous cell cancer, and verrucous; skin cancers including, but not limited to, basal cell carcinoma, squamous cell carcinoma and melanoma, superficial spreading melanoma, nodular melanoma, lentigo malignant melanoma, acral lentiginous melanoma; kidney cancers including, but not limited to, renal cell cancer, adenocarcinoma, hypernephroma, fibrosarcoma, transitional cell cancer (renal pelvis and/or uterer); Wilms' tumor; bladder cancers including, but not limited to, transitional cell carcinoma, squamous cell cancer, adenocarcinoma, carcinosarcoma. In addition, cancers include myxosarcoma, osteogenic sarcoma, endotheliosarcoma, lymphangioendotheliosarcoma, mesothelioma, synovioma, hemangioblastoma, epithelial carcinoma, cystadenocarcinoma, bronchogenic carcinoma, sweat gland carcinoma, sebaceous gland carcinoma, papillary carcinoma and papillary adenocarcinomas (for a review of such disorders, see Fishman et al., 1985, Medicine, 2d Ed., J.B. Lippincott Co., Philadelphia and Murphy et al., 1997, Informed Decisions: The Complete Book of Cancer Diagnosis, Treatment, and Recovery, Viking Penguin, Penguin Books U.S.A., Inc., United States of America).

b. Infectious Disease

The disclosed compositions and methods can be used to treat infections and infectious diseases. Generally, the agents are used to stimulate or enhance an immune response to infection in the subject by administering to the subject an amount of an activity sensing immunotherapeutic agent that modulates immune checkpoint molecule expression, ligand binding, crosslinking, suppressive signaling, or a combination thereof. In one embodiment, the immunotherapeutic agent inhibits, reduces, or blocks a suppressive immune signal transduction through the immune checkpoint molecule. In another embodiment, the immunotherapeutic agent induces, promotes, or enhances an immune response by inducing, promoting, or enhancing signal transduction through an immune checkpoint molecule. The method can reduce one or more symptoms of the infection.

The infection or disease can be caused by a bacterium, virus, protozoan, helminth, or other microbial pathogen that enters intracellularly and is attacked, i.e., by cytotoxic T lymphocytes.

The infection or disease can be acute or chronic. An acute infection is typically an infection of short duration. During an acute microbial infection, immune cells begin expressing immunomodulatory receptors. Accordingly, in some embodiments, the method includes increasing an immune stimulatory response against an acute infection.

The infection can be caused by, for example, but not limited to Candida albicans, Listeria monocytogenes, Streptococcus pyogenes, Streptococcus pneumoniae, Neisseria meningitidis, Staphylococcus aureus, Escherichia coli, Acinetobacter baumannii, Pseudomonas aeruginosa or Mycobacterium.

In some embodiments, the disclosed compositions are used to treat chronic infections, for example infections in which T cell exhaustion or T cell anergy has occurred causing the infection to remain with the host over a prolonged period of time.

Exemplary infections to be treated are chronic infections cause by a hepatitis virus, a human immunodeficiency virus (HIV), a human T-lymphotrophic virus (HTLV), a herpes virus, an Epstein-Barr virus, or a human papilloma virus.

Because viral infections are cleared primarily by T cells, an increase in T-cell activity would be therapeutically useful in situations where more rapid or thorough clearance of an infective viral agent would be beneficial to an animal or human subject. Thus, the disclosed compositions can be administered for the treatment of local or systemic viral infections, including, but not limited to, immunodeficiency (e.g., HIV), papilloma (e.g., HPV), herpes (e.g., HSV), encephalitis, influenza (e.g., human influenza virus A), and common cold (e.g., human rhinovirus) and other viral infections, caused by, for example, HTLV, hepatitis virus, respiratory syncytial virus, vaccinia virus, and rabies virus. The molecules can be administered topically to treat viral skin diseases such as herpes lesions or shingles, or genital warts. The molecules can also be administered systemically to treat systemic viral diseases, including, but not limited to, AIDS, influenza, the common cold, or encephalitis.

Representative infections that can be treated, include but are not limited to infections cause by microorganisms including, but not limited to, Actinomyces, Anabaena, Bacillus, Bacteroides, Bdellovibrio, Bordetella, Borrelia, Campylobacter, Caulobacter, Chlamydia, Chlorobium, Chromatium, Clostridium, Corynebacterium, Cytophaga, Deinococcus, Escherichia, Francisella, Halobacterium, Heliobacter, Haemophilus, Hemophilus influenza type B (HIB), Hyphomicrobium, Legionella, Leptspirosis, Listeria, Meningococcus A, B and C, Methanobacterium, Micrococcus, Myobacterium, Mycoplasma, Myxococcus, Neisseria, Nitrobacter, Oscillatoria, Prochloron, Proteus, Pseudomonas, Phodospirillum, Rickettsia, Salmonella, Shigella, Spirillum, Spirochaeta, Staphylococcus, Streptococcus, Streptomyces, Sulfolobus, Thermoplasma, Thiobacillus, and Treponema, Vibrio, Yersinia, Cryptococcus neoformans, Histoplasma capsulatum, Candida albicans, Candida tropicalis, Nocardia asteroides, Rickettsia ricketsii, Rickettsia typhi, Mycoplasma pneumoniae, Chlamydial psittaci, Chlamydial trachomatis, Plasmodium falciparum, Trypanosoma brucei, Entamoeba histolytica, Toxoplasma gondii, Trichomonas vaginalis and Schistosoma mansoni.

Other microorganisms that can be treated using the disclosed compositions and methods include, bacteria, such as those of Klebsiella, Serratia, Pasteurella; pathogens associated with cholera, tetanus, botulism, anthrax, plague, and Lyme disease; or fungal or parasitic pathogens, such as Candida (albicans, krusei, glabrata, tropicalis, etc.), Cryptococcus, Aspergillus (fumigatus, niger, etc.), Genus Mucorales (mucor, absidia, rhizophus), Sporothrix (schenkii), Blastomyces (dermatitidis), Paracoccidioides (brasiliensis), Coccidioides (immitis) and Histoplasma (capsulatuma), Entamoeba, histolytica, Balantidium coli, Naegleria fowleri, Acanthamoeba sp., Giardia Zambia, Cryptosporidium sp., Pneumocystis carinii, Plasmodium vivax, Babesia microti, Trypanosoma brucei, Trypanosoma cruzi, Toxoplasma gondi, etc.), Sporothrix, Blastomyces, Paracoccidioides, Coccidioides, Histoplasma, Entamoeba, Histolytica, Balantidium, Naegleria, Acanthamoeba, Giardia, Cryptosporidium, Pneumocystis, Plasmodium, Babesia, or Trypanosoma, etc.

c. Transplant Rejection

In another embodiment, the disclosed compositions and methods can be used prophylactically or therapeutically to reduce or inhibit graft rejection or graft verse host disease. Transplant rejection occurs when a transplanted organ or tissue is not accepted by the body of the transplant recipient. Typically rejection occurs because the immune system of the recipient attacks the transplanted organ or tissue. The disclosed methods can be used to promote immune tolerance of the transplant or graft by the recipient by administering to the subject an effective amount of one or more of the disclosed activity sensing immunotherapeutic agents. In one embodiment, the induction of immune tolerance can be measured by analyzing the amount of detectable molecule that is released in the urine of the subject receiving the immunotherapeutic agent for the reduction or inhibition of transplant rejection.

The transplanted material can be cells, tissues, organs, limbs, digits or a portion of the body, for example the human body. The transplants are typically allogenic or xenogenic. The disclosed compositions are administered to a subject in an effective amount to reduce or inhibit transplant rejection. The compositions can be administered systemically or locally by any acceptable route of administration. In some embodiments, the compositions are administered to a site of transplantation prior to, at the time of, or following transplantation. In one embodiment, compositions are administered to a site of transplantation parenterally, such as by subcutaneous injection.

In other embodiments, the compositions are administered directly to cells, tissue or organ to be transplanted ex vivo. In one embodiment, the transplant material is contacted with the compositions prior to transplantation, after transplantation, or both.

In other embodiments, the compositions are administered to immune tissues or organs, such as lymph nodes or the spleen.

The transplant material can also be treated with enzymes or other materials that remove cell Surface proteins, carbohydrates, or lipids that are known or suspected of being involved with immune responses such as transplant rejection.

i. Cells

Populations of any types of cells can be transplanted into a subject. The cells can be homogenous or heterogeneous. Heterogeneous means the cell population contains more than one type of cell. Exemplary cells include progenitor cells such as stem cells and pluripotent cells which can be harvested from a donor and transplanted into a subject. The cells are optionally treated prior to transplantation as mentioned above.

ii. Tissues

Any tissue can be used as a transplant. Exemplary tissues include skin, adipose tissue, cardiovascular tissue such as veins, arteries, capillaries, valves; neural tissue, bone marrow, pulmonary tissue, ocular tissue such as corneas and lens, cartilage, bone, and mucosal tissue.

iii. Organs

Exemplary organs that can be used for transplant include but are not limited to kidney, liver, heart, spleen, bladder, lung, stomach, eye, tongue, pancreas, intestine, etc. The organ to be transplanted can also be modified prior to transplantation as discussed above.

One embodiment provides a method of inhibiting or reducing chronic transplant rejection in a subject by administering an effective amount of the composition to inhibit or reduce chronic transplant rejection relative to a control.

iv. Graft Versus Host Disease (GVHD)

The disclosed compositions and methods can be used to treat graft-versus-host disease (GVHD) by administering an effective amount of the composition to alleviate one or more symptoms associated with GVHD. GVHD is a major complication associated with allogeneic hematopoietic stem cell transplantation in which functional immune cells in the transplanted marrow recognize the recipient as “foreign’ and mount an immunologic attack. It can also take place in a blood transfusion under certain circumstances. Symptoms of GVHD include skin rash or change in skin color or texture, diarrhea, nausea, abnormal liver function, yellowing of the skin, increased susceptibility to infection, dry, irritated eyes, and sensitive or dry mouth.

d. Autoimmunity and Chronic Infection

The disclosed immunotherapeutic agents can also be used to treat inflammatory or autoimmune diseases and disorders. In such an embodiment, the immunotherapeutic agent is one that modulates stimulatory immune checkpoint molecule expression, ligand binding, crosslinking, suppressive signaling, or a combination thereof. Representative inflammatory or autoimmune diseases/disorders include, but are not limited to, rheumatoid arthritis, systemic lupus erythematosus, alopecia areata, ankylosing spondylitis, antiphospholipid syndrome, autoimmune Addison's disease, autoimmune hemolytic anemia, autoimmune hepatitis, autoimmune inner ear disease, autoimmune lymphoproliferative syndrome (alps), autoimmune thrombocytopenic purpura (ATP), Behcet's disease, bullous pemphigoid, cardiomyopathy, celiac sprue-dermatitis, chronic fatigue syndrome immune deficiency, syndrome (CFIDS), chronic inflammatory demyelinating polyneuropathy, cicatricial pemphigoid, cold agglutinin disease, Crest syndrome, Crohn's disease, Dego's disease, dermatomyositis, dermatomyositis—juvenile, discoid lupus, essential mixed cryoglobulinemia, fibromyalgia—fibromyositis, grave's disease, guillain-barre, hashimoto's thyroiditis, idiopathic pulmonary fibrosis, idiopathic thrombocytopenia purpura (ITP), Iga nephropathy, insulin dependent diabetes (Type I), juvenile arthritis, Meniere's disease, mixed connective tissue disease, multiple sclerosis, myasthenia gravis, pemphigus vulgaris, pernicious anemia, polyarteritis nodosa, polychondritis, polyglancular syndromes, polymyalgia rheumatica, polymyositis and dermatomyositis, primary agammaglobulinemia, primary biliary cirrhosis, psoriasis, Raynaud's phenomenon, Reiter's syndrome, rheumatic fever, sarcoidosis, scleroderma, Sjogren's syndrome, stiff-man syndrome, Takayasu arteritis, temporal arteritis/giant cell arteritis, ulcerative colitis, uveitis, vasculitis, vitiligo, and Wegener's granulomatosis.

In some embodiments the inflammation or autoimmune disease is caused by a pathogen, or is the result of an infection.

### EXAMPLES

### Example 1. Checkpoint Blockade Immunotherapy Agents Modified with Protease Substrates Retain Target Binding and Sense Granzyme B Activity

Materials and Methods:

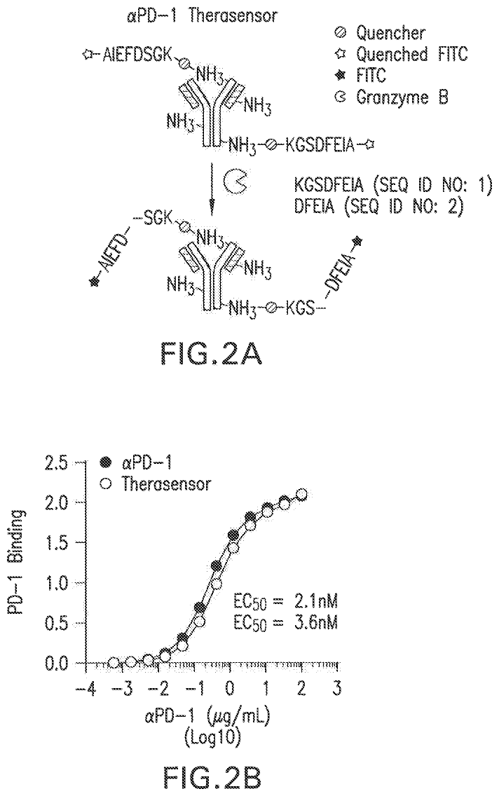

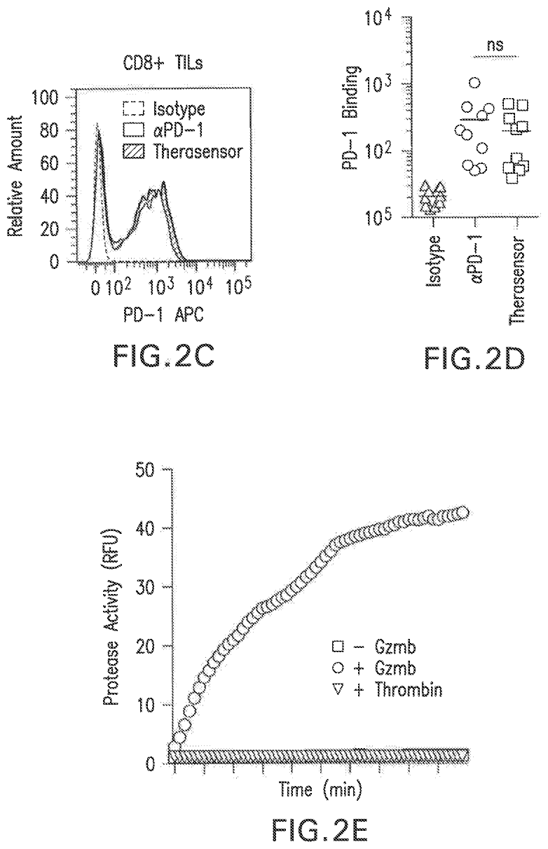

To combine therapeutic activity and response monitoring capability, αPD-1 cancer immunotherapy antibodies were functionalized with granzyme B (GzmB) protease sensing biomarkers using amine reactive chemistry (FIG. 2A).

Results:

αPD-1 maintained targeting ability when functionalized with a GzmB protease substrate as determined by a similar EC50 benchmarked against unmodified αPD-1 (FIG. 2B). Functionalized αPD-1 also retained target binding to tumor infiltrating CD8+ T cells (FIG. 2C).

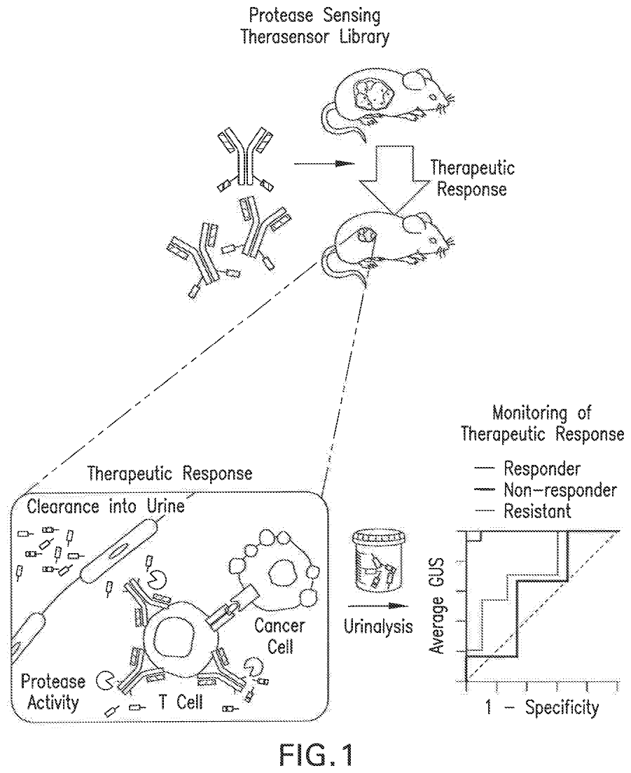

To determine if GzmB could access and cleave antibody-conjugated substrates, αPD-1 was functionalized with GzmB substrate engineered with a quencher molecule before the cleavage site and a fluorescent reporter (FAM) after (FIG. 1). Following cleavage, the reporter is separated from the quencher, producing a fluorescent signal for quantitation. Using an in vitro cleavage assay, functionalized αPD-1 demonstrated specific cleavage by GzmB, with no cross-cleavage by thrombin, a common serum protease (FIG. 2E).

### Example 2. Functionalized Co-Stimulation Blockade Therapeutics are Functional and Sense Granzyme B Activity

Materials and Methods:

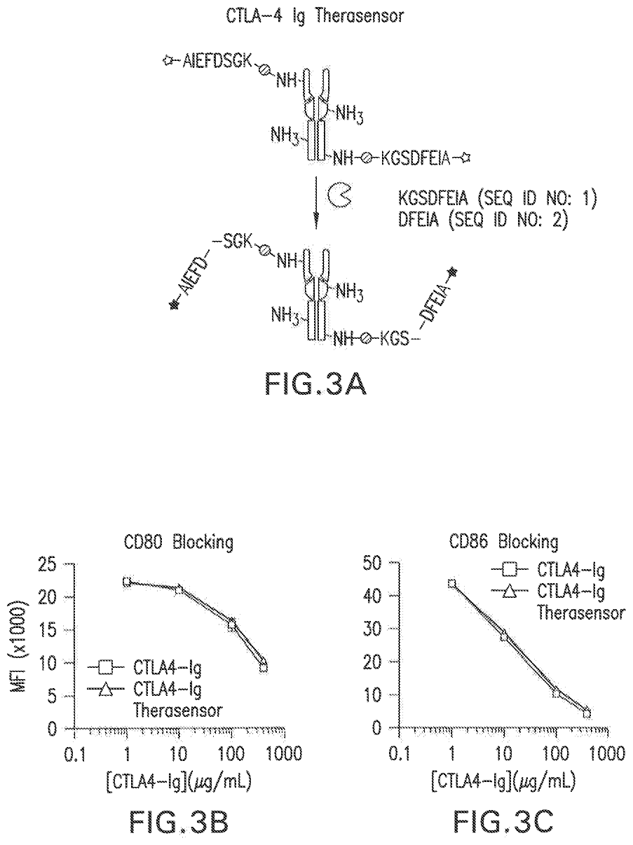

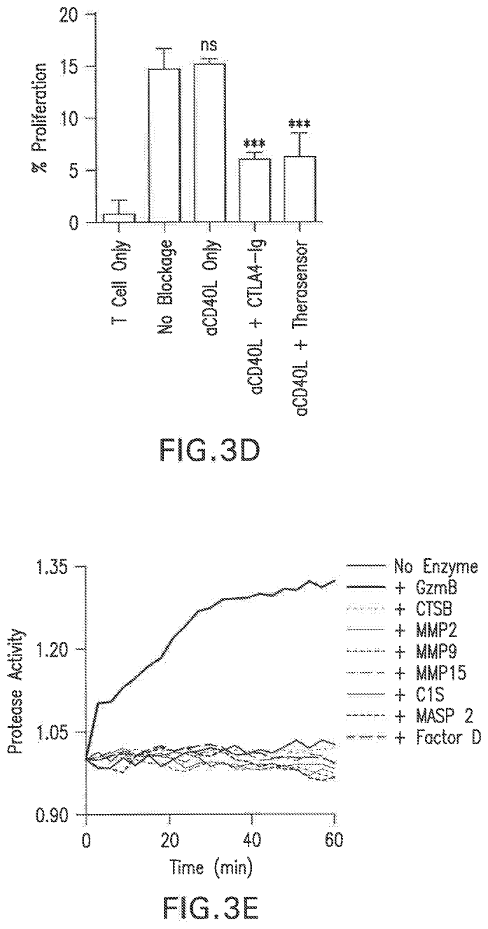

To determine the applicability of this approach to other protein biologics, abatacept, a CTLA-4 Ig fusion protein that binds to CD80 and CD86 to block T cell co-stimulation, was functionalized with GzmB substrate as described above and in FIG. 3A.

Results:

Functionalized CTLA-4 Ig targeted to CD80/CD86 with similar efficacy to unmodified CTLA-4 Ig, as determined by competitive binding with anti-CD80 and CD86 antibodies (FIGS. 3B-3C). Functionalization with GzmB protease substrates did not compromise the ability of CTLA-4 to dampen T cell activation and proliferation when benchmarked against unmodified protein (FIG. 3D). Using an in vitro cleavage assay, modified CTLA-4 Ig demonstrated specific cleavage by GzmB, with no cross-cleavage by matrix, complement, or immune proteases (FIG. 3E). Combined, this demonstrates that orthogonal immunotherapeutic agents (αPD-1 and CTLA-4 Ig) can be functionalized with protease sensing substrates without loss of function.

### Example 3. Immunotherapeutic Agents Functionalized with GzmB Sense CD8+ T Cell Mediated Cytotoxicity

Materials and Methods:

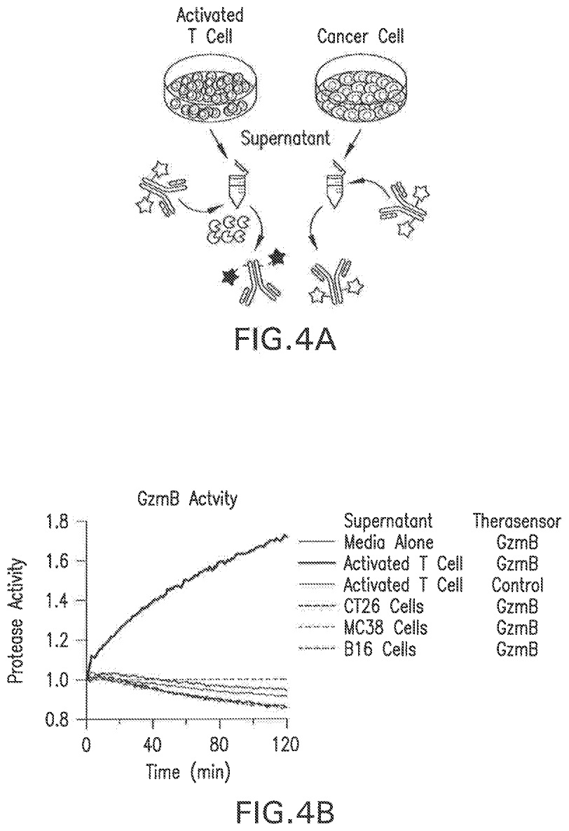

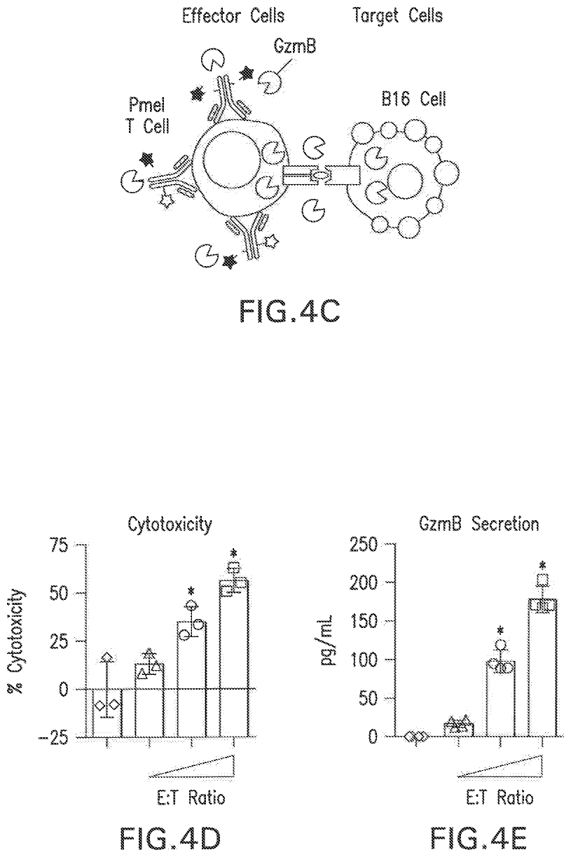

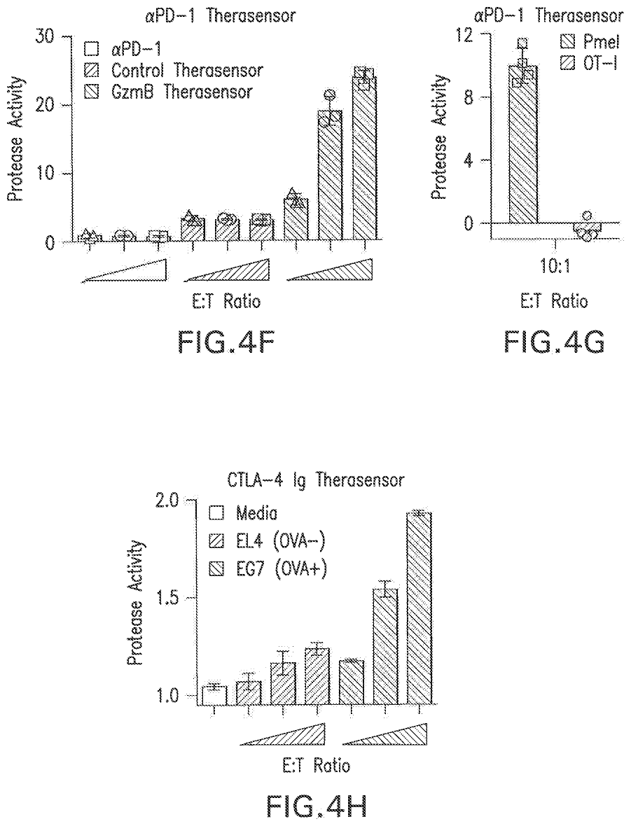

To determine the ability of functionalized immunotherapeutic agents to detect T cell activity, αPD-1 functionalized with GzmB substrate was incubated with supernatant isolated from activated CD8+ T cells or various cancer cell lines (CT26, MC38, or B16 cell lines) (FIG. 4A).

Results:

The functionalized αPD-1 was not cleaved when incubated with supernatants from any of the cancer cell lines but displayed increased fluorescent signal over time when incubated with activated T cell supernatants (FIG. 4B). Control αPD-1 conjugated to a control substrate (LQRIYK, (SEQ ID NO:3)) for complement protease C1s was also not cleaved by activated T cell supernatants. GzmB activity sensing was tested during co-incubation of CD8+ T cells isolated from the Pmel-1 TCR transgenic mouse (gp100 specific) and B16 melanoma cells (expresses gp100 and are recognized by Pmel T cells) (FIG. 4C) (Klebanoff, et al., Clin Cancer Res, 17(16):5343-5352 (2011); Abad, et al., J Immunother, 31(1):1-6 (2008); Overwijk, et al., J Exp Med, 198(4):569-580 (2003)). Addition of functionalized αPD-1, but not control αPD-1, resulted in significantly increased fluorescence signals across multiple T cell to target cell ratios, corresponding with increased cell killing and GzmB protein secretion (FIGS. 4D-4F). Increased signal was not observed when co-culturing with OT-I T cells, which do not recognize B16 cells, verifying the protease activity measured corresponded with antigen-specific T cell mediated cellular cytotoxicity (FIG. 4G). Cleavage of GzmB substrate functionalized CTLA-4 Ig was tested using a transgenic OT-I T cell system, which recognize the peptide epitope SIINFEKL (SEQ ID NO:4) from chicken ovalbumin (OVA) and target OVA expressing EG7 cells, but not the parental EL4 cell line that lacks OVA expression. Incubation of OT-I T cells with EG7-OVA cells, but not EL4 control cells, resulted in increased fluorescent signaling (FIG. 4H). Combined, this demonstrates that immunotherapeutic agents (αPD-1 and CTLA-4 Ig) functionalized with protease sensing substrates can sense T cell activity and specifically sense cytotoxicity.

### Example 4. Granzyme B Protease Activity Corresponds with Responsive Immunotherapy

Results:

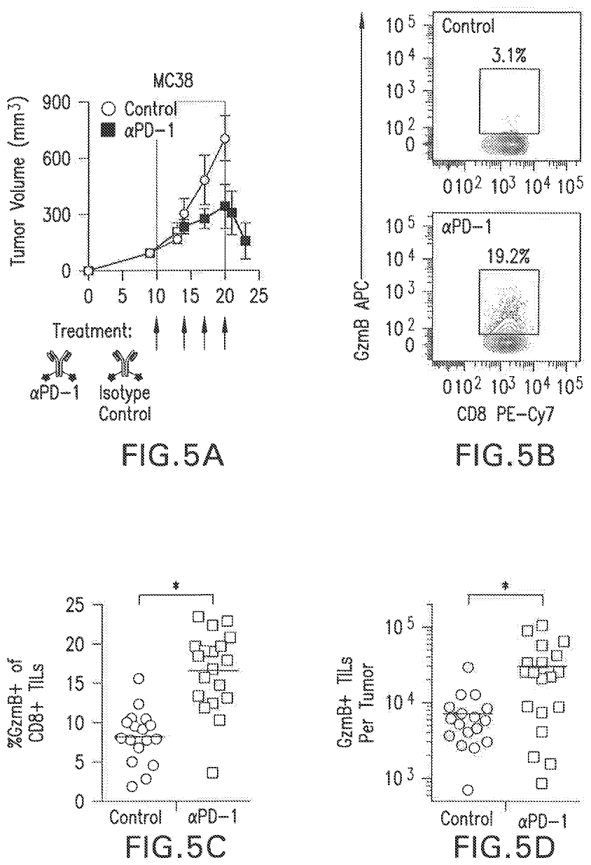

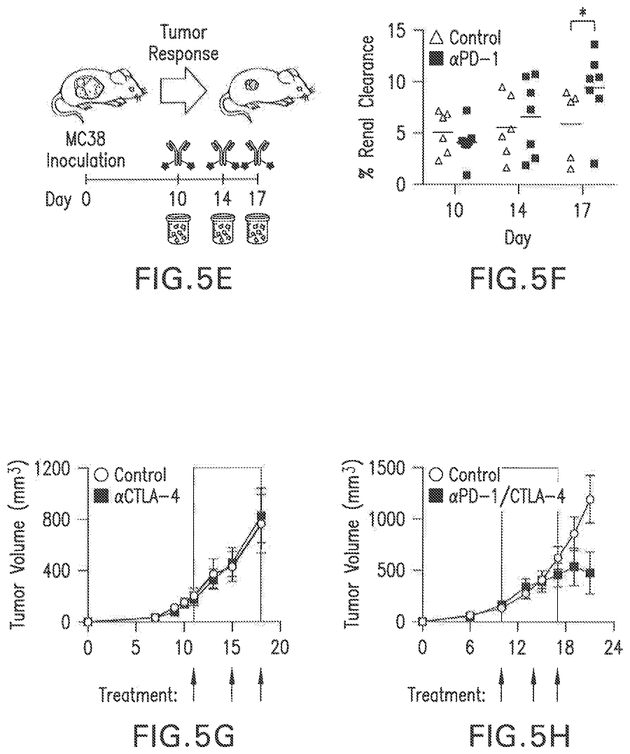

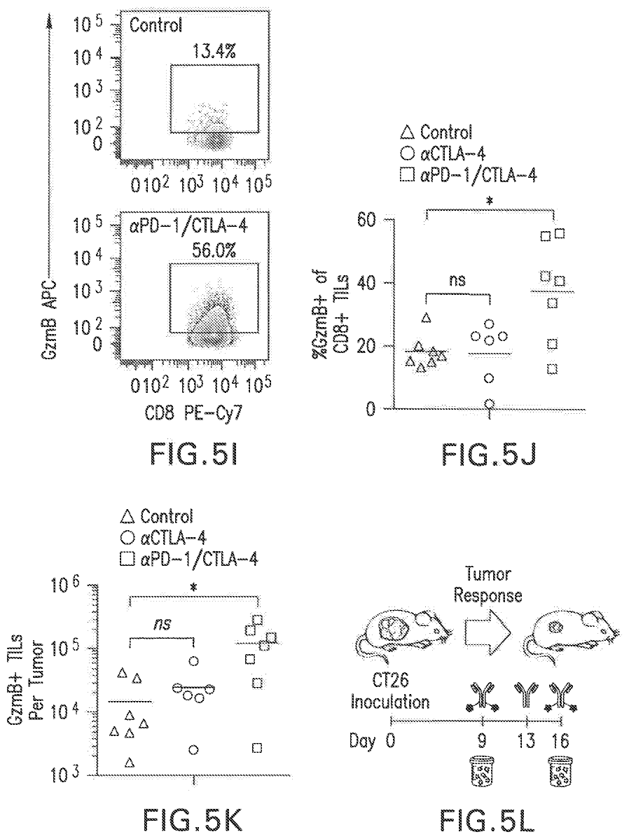

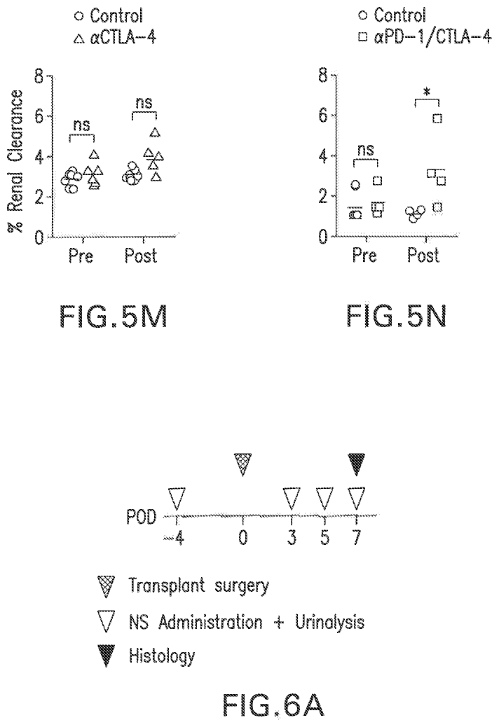

To determine the importance of protease activity as a biomarker of responsive immunotherapy, GzmB protease expression kinetics were defined within tumor infiltrating CD8+ T cells during immunotherapy treatment in the PD-1 responsive MC38 tumor model (FIG. 5A). Responsive immunotherapy during PD-1 blockade corresponded with increased numbers of CD8+ TILs expressing the cytotoxic mediator GzmB (FIGS. 5B-5D). MC38 mice were next treated with αPD-1 or isotype control functionalized with GzmB substrate, allowing for quantification of protease activity before (day 11) and during early treatment (day 14 and 17) (FIGS. 5E-5F). Responsive therapy correlated with increased GzmB activity as determined by increased urine signal on Day 17 in the αPD-1, but not isotype control, treated mice. Using a CT26 tumor model, GzmB expression within CD8+ T cells and activity, as detected by urine secretion of cleaved biomarkers, was also increased early in treatment during responsive αPD-1/CTLA-4 combined therapy, but not during non-responsive αCTLA-4 monotherapy (FIG. 5G-5N). Combined, these data demonstrate that GzmB protease activity can serve as a biomarker for early treatment response to immunotherapy. Future development of the technology will identify protease signatures that correspond to responsive immunotherapy to inform building of a multiplex biomarker library, including GzmB and other top enriched immune and disease specific proteases.

### Example 5. CD8 T Cell Accumulation and Expression of Granzyme B Protease at the Graft Site Corresponds with the Onset of Acute Cellular Rejection

Results:

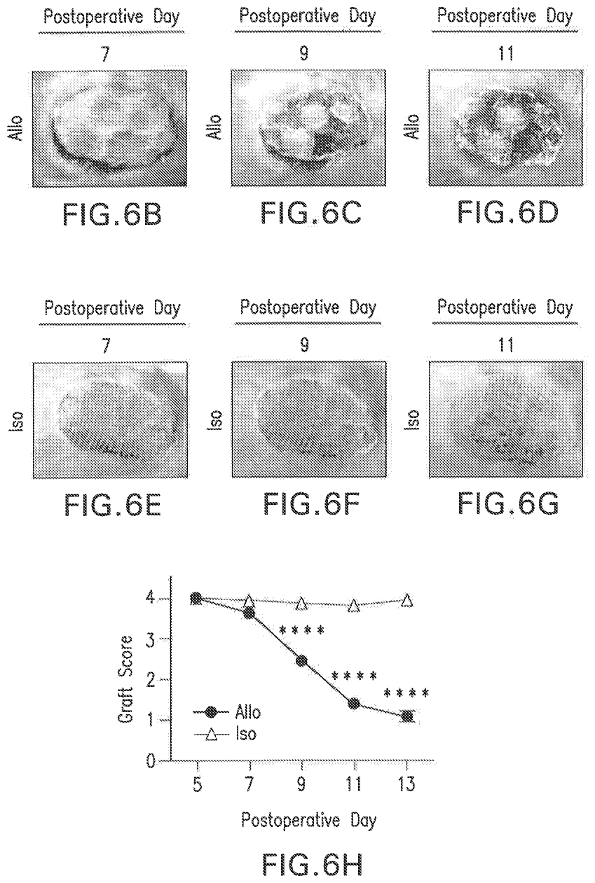

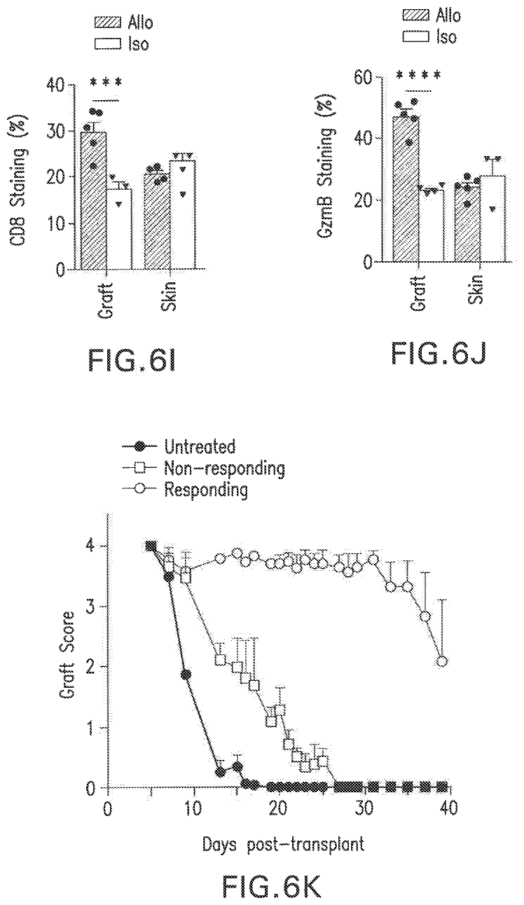

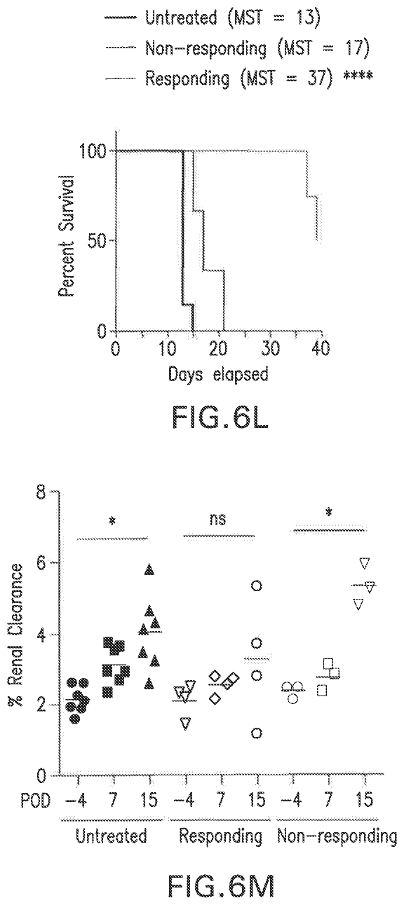

Histological criteria for staging severity of ACR include features, such as tissue damage and presence of apoptotic cells, which are downstream effects of anti-graft T cell responses. Activity measurements of proteases that drive disease pathology have the potential to be early biomarkers and anticipate disease trajectory, such as using MMP activity to predict liver fibrosis progression and regression. Therefore the potential of using GzmB activity nanosensors, which consists of an iron oxide nanoparticle core (IONP) conjugated with GzmB protease substrates, for early detection of ACR was investigated (FIG. 6A). To quantify skin graft health and rejection kinetics, a score of 4 was assigned for healthy allografts, a score of 0 was assigned for full rejection, and intermediate scores were assigned based on features such as the ratio of viable to necrotic skin and the presence of ulcerations or scabs. According to these metrics, graft scores began to significantly decrease at day 9 after transplant and reached endpoint when allografts were completely rejected within two weeks post-transplant (FIGS. 6B-6H). To identify the earliest timepoint of GzmB upregulation, graft tissue was analyzed on day 7 by immunohistochemistry and significant increases were found in both graft-infiltrating CD8 T cells and GzmB expression levels (FIGS. 6I-6J). Taken together, this data provides evidence that GzmB expression and activity are significantly upregulated in allograft tissue at the onset of acute cellular rejection.

### Example 6. Responding and Non-Responding CTLA-4 Ig Treatment Groups can be Stratified by Granzyme B Protease Activity

Results:

Abatacept, a CTLA-4 Ig fusion protein that binds to CD80 and CD86 to block T cell co-stimulation, is used in the clinic to prevent rejection of transplanted organs and for treating various chronic inflammatory and autoimmune diseases. A co-stimulation blockade therapeutic model was developed where skin graft recipient mice (BALB/c skin to BL/6 recipient mice) were treated with CTLA-4 Ig and monitored for graft health and survival. CTLA-4 Ig treatment prolonged graft survival in a subset of animals (“responding”), while other mice remained non-responsive to treatment and ultimately rejected the graft (“non-responding”) at a rate similar to untreated animals (FIGS. 6K-6L). Using GzmB functionalized CTLA-4 Ig, significantly increased GzmB activity was observed POD 15 in untreated and CTLa-4 Ig non-responding groups, but not in CTLA-4 responding groups, corresponding with prolonged graft survival (FIG. 6M).

### Example 7. Tumor Protease Signatures of ICB Response and Acquired Resistance

Results:

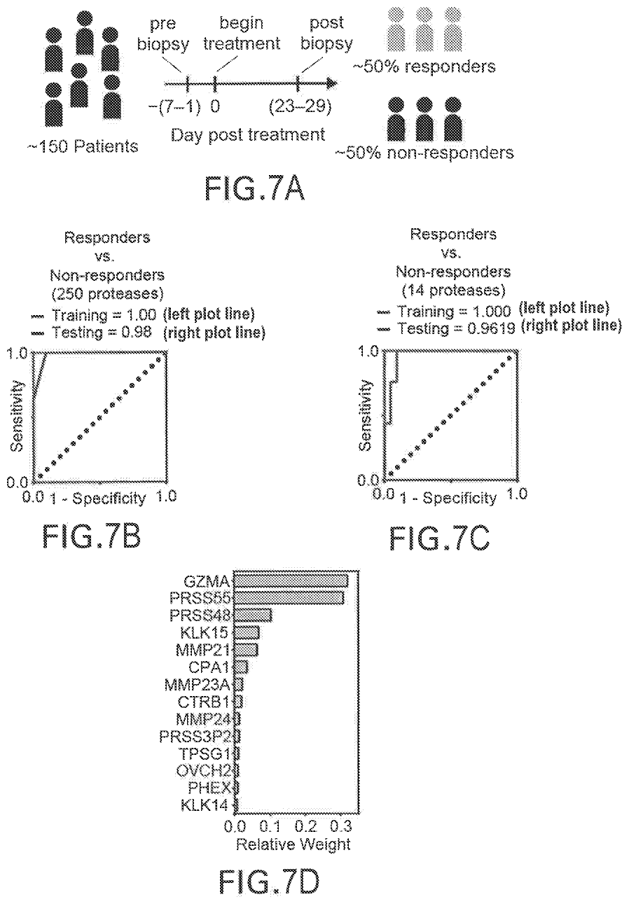

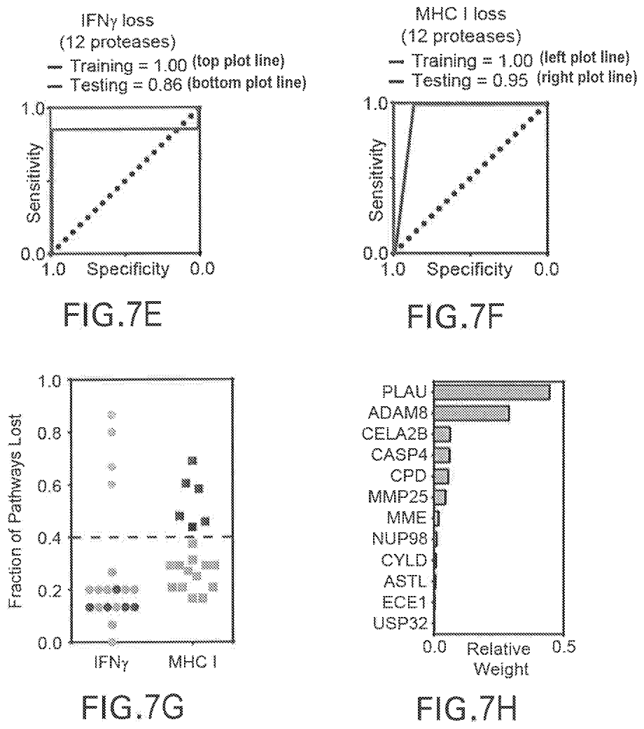

A significant fraction of patients who have objective responses will eventually relapse despite continued checkpoint inhibitor treatment (e.g., up to one third for melanoma). Mechanisms of resistance include impaired T cell recognition (loss of antigen-presenting machinery) or activity (insensitivity to IFN-γ signaling). To identify changes in protease expression during checkpoint inhibitor response and resistance, an independent study (Hugo, et al., Cell, 165:35-44 (2016); Riaz, et al., Cell, 171:934-949 (2017)) of serial biopsies of 68 melanoma patients before and early-on-treatment with αPD-1 was studied (FIG. 7A). The expression levels of 250 extracellular proteases were used as features to classify responders from non-responders with a binary classifier by Support Vector Machine (SVM) (FIG. 7B). In equally split training and test validation cohorts, it was found that protease expression could be used to discriminate responders from non-responders with near-perfect AUROCs (>0.98). It was then asked of the 250 proteases which were the most important for classification, and by applying the Lasso algorithm, a shortened list of 14 key proteases was defined that could be used to classify the same patients with AUROC>0.96 (FIGS. 7C-7D). These results show that the expression of proteases can be used to classify patient responders from non-responders.

It was then determined whether proteases expression could be used to define mechanisms of resistance. Non-responding patient full gene transcripts were analyzed to find genes that were differentially expressed when compared to responding patients (genes with a tscore>100). With these genes, pathway analysis was run on frequent mechanisms of resistance to immune checkpoint therapies focused on two pathways in particular: IFNγ signaling and MHC I antigen presentation (FIGS. 7E-7F). Using this approach, a panel of proteases that can identify the mechanism of resistance as loss of sensitivity to IFNγ in non-responders was found, and a panel of proteases that can identify MHC I antigen presentation loss was also found (FIG. 7H). The fraction of pathways from each mechanism of resistance (IFNγ and MHC I presentation) showed that loss was represented in separate individual patients (FIG. 7G).

While in the foregoing specification this invention has been described in relation to certain embodiments thereof, and many details have been put forth for the purpose of illustration, it will be apparent to those skilled in the art that the invention is susceptible to additional embodiments and that certain of the details described herein can be varied considerably without departing from the basic principles of the invention.

All references cited herein are incorporated by reference in their entirety. The present invention may be embodied in other specific forms without departing from the spirit or essential attributes thereof and, accordingly, reference should be made to the appended claims, rather than to the foregoing specification, as indicating the scope of the invention.

We claim:

1. A method of administering and monitoring responses to immunotherapy in a subject in need thereof, comprising:
administering to the subject an effective amount of at least one therapeutic agent linked to protease substrate that provides a detectable signal in response to protease activity promoted by the therapeutic agent;
detecting and measuring the signal in a sample from the subject;
determining an effect of the therapeutic agent on the subject, wherein the subject is determined to be responsive to the therapeutic agent if the detectable signal is detected, and the subject is determined to be non-responsive to the therapeutic agent if the detectable signal is not detected; and
administering the same effective amount of the therapeutic agent to responsive subjects, or adjusting the effective amount of therapeutic agent administered to non-responsive subjects.

2. The method of claim 1, wherein the therapeutic agent is an immune checkpoint inhibitor.

3. The method of claim 2, wherein the immune checkpoint inhibitor is an anti-PD-1 or anti-CTLA-4 antibody.

4. The method of claim 1, wherein the therapeutic agent is an immunosuppressive agent.

5. The method of claim 4, wherein the immunosuppressive agent is CTLA-4 Ig.

6. The method of claim 1, wherein the protease substrate is conjugated to a reporter molecule.

7. The method of claim 6, wherein the reporter molecule is a fluorescent molecule, a bioluminescent molecule, or a mass-tag.

8. The method of claim 1, wherein the protease substrate comprises a quencher molecule and a fluorescent molecule flanking the substrate.

9. The method of claim 1, wherein the detectable signal is a peptide fragment from the protease substrate.

10. The method of claim 1, wherein the detectable signal is a fluorescent reporter.

11. The method of claim 1, wherein the detectable signal is a mass-tag.

12. The method of claim 1, wherein adjusting the effective amount of immunotherapeutic agent additionally comprises administering a different immunotherapeutic agent.

13. The method of claim 1, wherein the sample comprises a urine sample or a blood sample.

14. The method of claim 1, wherein measuring the signal comprises subjecting the sample to mass spectrometry, flow cytometry, or ELISA.

15. The method of claim 1, wherein the non-responsive subject has immune resistance.

16. The method of claim 1, wherein the subject has cancer.

17. The method of claim 1, wherein the subject has an infectious disease.

18. The method of claim 1, wherein the subject has a transplanted organ.

19. A composition comprising, a therapeutic agent conjugated to a protease substrate that provides a detectable signal in response to protease activity promoted by the therapeutic agent.

20. The composition of claim 19, wherein the therapeutic agent is an immune checkpoint inhibitor.

21. The composition of claim 20, wherein the immune checkpoint inhibitor is an anti-PD1 or anti-CTLA4 antibody.

22. The composition of claim 19, wherein the therapeutic agent is an immunosuppressive agent.

23. The composition of claim 22, wherein the immunosuppressive agent is CTLA-4 Ig.

24. The composition of claim 19, wherein the detectable signal is a peptide fragment from the protease substrate.

25. The composition of claim 19, wherein the protease substrate is conjugated to a reporter molecule.

26. The method of claim 25, wherein the reporter molecule is a fluorescent molecule, a bioluminescent molecule, or a mass-tag.

27. The method of claim 19, wherein the protease substrate comprises a quencher molecule and a fluorescent molecule flanking the substrate.
